SEQUENCE LISTING (5 sequences)

SEQ ID NO: 1 - DNA, 1001 bp, Homo sapiens
ggacagcccctctttaaggaacatctcccgggccgagcgttctgagcgccctctgagcaggcagcccaccaccggggcgctggccagtctgagtcagtcctgcctgggaggcagtcaactctggggaaggcagaagaatcacagcactgagtgccgtccagtttcccctcaactatattcgcggacacggcgccaacaggggaaaacttacatagcacctattcattctgaggcctggccggcctctggaaatcagcatttcagtgttggccaggaaggaggccagaactgtgctgtagcatttcaatgtatggctgtcccacaatttatattccagtcccctgatgatggacatgtaggcggtttccagtttttttcatgatcacagatcacacttctgtgaacagctctggatgtgtctccttgtgcaaatatggtgctaggtgttcttcagagcatttttctccaaacttgccgggtaatatggtcaccttggggagyttgttaaatatacatatttccaggtctgctctggtgggtctgatatttggagccaggaatcttttctttcttttaacagatacttaagtgattcctgtggtccaggaagttttgcaagctctgatgcagaagagctcgctggagcaaacatgtatgttctgttttactaattagatatacgctgttgatgactccaaagtggacgtgccatttctgtcccctccagcagtaggaaagaatcccattatcccacatcctggccagttcttggtattgtgagatttttttaaacttcttctcttaagactatgatggtattttgttgtaatataatccacattttctatattattactgtgcttgagcacttcttcaaatgtttgatgaccttgaccattgaaacttcctttttgtgaattacctattcatatcctttgtagcttcactgtggaatgcttgttactgatttgtagccattctttatacatttcactcccact

SEQ ID NO: 2 - DNA, 121 bp, Homo sapiens
acagttggtgacattattcagtttgacatctgacatatttaaaagcaattctagcaacttyggggaaaaaaaaataactcttaccaggaaataactagaaaaatcagcaaggtggaacaag

SEQ ID NO: 3 - DNA, 1001 bp, Homo sapiens
cactgaagtgaaagatgagttaacaatgacaaggtacagagattataatatagatgagggagagagaaactcggcctgaggaggtcaggaaaggtattttagagaaactgatttcactatataaatgttgtattaacacaaatcttactttgttatggattcagactgctgacagggcaacagcattatctccctaaagaatgagaaattcattccatagcaaatttattagaagagagtctaaaatgtcctaatactaccagtgactcctctaggaaaaaaattgtcatataatttagttatttctaaagcagtttgaaagtagcttggcctaaagctctgattatattaattttttaaagaaacaattattcattcactgtatgaggattattattatttgtctcatgttgtgtttgcatatccatgagagttagatgagtcattttcttttgttttactttttaatacattagcaaattataaaattactcatattayaccacaaagattacaaggatggcagctttggccagtgtagtagtcccacctattgattagagtcaaaagtaaagcccagccctgctttgtgcattgctcctaataaagtggatgttacttaacacatacgcagaagacagaagcgtcttcgtgtcctcactttactcctcactttcttaactgcttaagtatttccacgatataaatgcagtgataataataatacggacagtccctgacttaacgatttttcaacttttatgatggtgggaaagtgatacgcattcagtatggctcctcgacttacaatggggttgcctccagataaacccattgtgaattgaaaatatcttacacttagcactccattcttaatacctgctagaattatagattatccctcaaaattggcatagtataatatgggtatcagcaagttgttgcactttattcagagctttacactaggcaggggtgggctttacttttgactctaatca

SEQ ID NO: 4 - DNA, 1001 bp, Homo sapiens
ttagtagagacagggtttcaccatgttggccaggctggtcttgaactcctgacctcgtgatccgcccggcttggcctcccaaagtgctggggttacagacatgggccaccacacccggccaaaaggcgatattttttaaaacacatcaattgatttaccaaatatatatttttaaacttttaggcttggggatacatgtgagggttcgttaaataggtaaacacatgtcacgggggcttgttgtacatattatttcatcacccagatattaggcccagtacccaatagttgtatcttttctgctcctctccctcctcccaccctcagtaccaggaattaaatatgaataatgaaggtagctcagccaataaaactgaatcagttcttcagtctgtgggcaaacacaaaattcttctttcagaggtttgataagctgttgtagtttcgaaatgatccaaataataacttacatgacttgatttattaagaagcctgaaatayattaaacaaatcacagctccaaagaatgaacaataggataatggaatgttaaaggaaaacaaaccaatttaaaggaaatttccatctgcaaaaggcttagtaggatctgctaactcagaatcaggagtgttctttaatttatgaggttctcatttcagtactttaaaaaattaacggtaaatggccccatggcttatagcaaaaggagacattctatttaagcaaagttattagaacaatggtgattgtgagaagagctgaaaacaaaagtcagtcactcagcaactagaatgccagaaagaaaaaagaaaagacaaaaatagaaatacatctaccactggtcctttcaggacatctggatacatggctcataactaccccaagtaacatccctttcaaacacaagaagagaagtcttctaactgccaaaactgttctaacagaacatgtgtatcatatcctaattagtattagcatattttttgaccaccagaaagttg

SEQ ID NO: 5 - protein, 9 aa, Homo sapiens
  C-term NH2
CYFQNCPRG

(19) United States
(12) Patent Application Publication
(10) Pub. No.: US 2013/0245038 A1
(43) Pub. Date: Sep. 19, 2013

(54) Method For Selecting Or Identifying A Subject For V1B Antagonist Therapy

(71) Applicants: ABBVIE DEUTSCHLAND GMBH & CO. KG, Wiesbaden (DE); ABBVIE INC., North Chicago, IL (US)

(72) Inventors: David Katz, Chicago, IL (US); Marcel van Gaalen, Wiesbaden (DE)

(73) Assignees: AbbVie Inc., North Chicago, IL (US); AbbVie Deutschland GmbH & Co. KG, Wiesbaden (DE)

(21) Appl. No.: 13/798,360

(22) Filed: Mar. 13, 2013

Related U.S. Application Data
(60) Provisional application No. 61/610,101, filed on Mar. 13, 2012.

(51) Int. Cl. G01N 33/68; C07D 401/14

(52) U.S. Cl. CPC G01N 33/6893; C07D 401/14
USPC 514/253.09; 436/501; 435/7.94; 435/6.11; 435/6.12

(57) ABSTRACT

Provided herein is a method for detecting an HPA axis function marker in a biological sample. The method may be used to determine whether a patient is a suitable candidate for treatment with a V_{1B} antagonist. The HPA marker may be a genomic marker, non-genomic marker, or a combination thereof. Depending on the type of HPA marker, the method of detection can be an immunoassay or genotyping, for example.

This application claims the benefit of U.S. Application No. 61/610,101 filed on Mar. 13, 2012, the contents of which are herein incorporated by reference.

### FIELD OF THE INVENTION

The present invention relates to methods for determining whether a subject is a suitable candidate for treatment with a V_{1B} antagonist.

### BACKGROUND

The role of vasopressin, or arginine vasopressin (AVP), in various pathological states has been the subject of intensive research in recent years and the selective antagonism of the various vasopressin receptors has opened up avenues to pursuing novel clinical prospects. Four receptors by which AVP mediates it effect are known: oxytocin, V_{1A}, V_{1B} or V₃, and V₂. Consistent with its location in the pituitary and central nervous system (CNS) limbic brain regions, an antagonist of the V_{1B} receptor shows anxiolytic and antidepressant effects in animal models. See Griebel et al., PNAS 99, 6370 (2002); and Serradeid-Le Gal et al., J. Pharm. Exp. Ther. 300, 1122 (2002). Although the particular CNS effects of vasopressin mediated by the V_{1B} receptor are not understood, the animal model effects appear to be mediated via both pituitary and CNS receptors.

AVP is released from the hypothalamus and is a key mediator of hypothalamus-pituitary-adrenal (HPA) axis activity. AVP acts at the pituitary gland via the V_{1B} receptor to stimulate release of adrenocorticotrophin hormone (ACTH), which in turn acts at the adrenal gland to simulate release of the stress hormone cortisol. By reducing levels of cortisol, a V_{1B} antagonist can be used to effectively treat disorders characterized by hypothalamic-pituitary adrenal axis (HPA axis) dysregulation.

Typically not all subjects who have a disorder respond similarly well to a therapy. Health care can be improved by methods that enable matching a subject with a therapy to which the subject is most likely to respond. A major disadvantage to overcome is that no method exists to identify a subject who is likely to respond favorably to treatment with a V_{1B} antagonist. Currently, the only means to select subjects for V_{1B} antagonist therapy is to observe the clinical course of disease during treatment. This potentially delays when a subject will receive effective treatment, thereby increasing morbidity risk and decreasing quality of life. Accordingly, there remains a need to identify one or more biological markers, genetic markers, or combinations thereof, that are associated with disorders related to response to a V_{1B} antagonist.

### SUMMARY OF THE INVENTION

In one aspect, the present invention is directed to a method for determining whether a subject is a suitable candidate for treatment with a V_{1B} antagonist. The method may comprise providing a biological sample from a subject and then detecting an HPA axis function marker in the sample. Alternatively, the method comprises detecting an HPA axis function marker in a biological sample obtained from the subject. The presence of the marker indicates that the subject is a suitable candidate for treatment with a V_{1B} antagonist. The HPA axis function marker being present at a level greater than about the 60^{th} percentile, preferably greater than about the 65^{th}, 70^{th}, 75^{th}, 80^{th}, 85^{th}, 90^{th}, or 95^{th} percentile, of the distribution of the marker in a normal subject sample indicates that the subject is a particularly suitable candidate for treatment with a V_{1B} antagonist. The subject may have a disorder characterized by HPA axis dysfunction.

The HPA axis function marker may be a nucleotide sequence comprising SEQ ID NO:1 (LHPP res7088418) and a nucleotide sequence comprising SEQ ID NO:2 (AKRID1 rs17169521); a nucleotide sequence comprising an NR3C1 genotype, or a combination thereof. The NR3C1 genotype may be SEQ ID NO:3 (rs10482672) or SEQ ID NO:4 (rs17100236). The marker may be detected by genotyping, such as amplifying the nucleic acid comprising the marker and then detecting the amplified nucleic acids, thereby detecting the marker. The marker may be detected by sequencing.

In another aspect, the present invention is directed to a method for determining whether a subject is a suitable candidate for treatment with a V_{1B} antagonist. The method may comprise providing a biological sample from a subject and then detecting an HPA axis function marker in the sample. If the HPA axis function marker is present at a level above about the 60^{th} percentile of the distribution of the marker in a normal subject sample, the subject is a suitable candidate for treatment with a V_{1B} antagonist. The subject may have a disorder characterized by HPA axis dysfunction. The HPA axis function marker may be present at a level greater than about the 65^{th}, 70^{th}, 75^{th}, 80^{th}, 85^{th}, 90^{th}, and 95^{th} percentile of the distribution of the HPA axis function marker in a normal subject sample. The marker may be AVP, copeptin, cortisol, cortisone, ACTH, a hepatic metabolite of cortisol, a hepatic metabolite of cortisone, CRH, or a combination thereof. The copeptin may be plasma copeptin. The AVP may be plasma AVP. The hepatic metabolite of cortisol may be alpha-tetrahydrocortisol, beta-tetrahydrocortisol, alpha-cortol, beta-cortol, alpha-cortolic acid, beta-cortolic acid, or a combination thereof. The hepatic metabolite of cortisone may be tetrahydrocortisone, cortolone, cortolonic acid, or a combination thereof. According to certain embodiments of the invention, the method for determining whether a subject is a suitable candidate for treatment with a V_{1B} antagonist described herein is an in vitro method. The biological sample may be a nucleic acid containing sample, serum, plasma, blood, urine or saliva. The biological sample may be urine. The marker may be the sum of urine amounts of cortisol, cortisone, alpha-tetrahydrocortisol, beta-tetrahydrocortisol, and tetrahydrocortisone. The marker may be the sum of urine amounts of alpha-tetrahydrocortisol, beta-tetrahydrocortisol, and tetrahydrocortisone. The marker may be the sum of all the cortisol and cortisone metabolites. The marker may be the sum of the urine amounts of cortisol and cortisone metabolites divided by the amount of creatinine in the same urine sample. The urine sample may be a 24-hour collection of urine from the subject. The urine sample may be an overnight collection of urine from the subject. The urine sample may be collected from the subject in a single void. The sample may be a plasma sample that contains greater than or equal to 9 pg/mL AVP as determined by radioimmunoassay. The sample may be a plasma sample that contains greater than or equal to 2.75 ng/mL of copeptin as determined by enzyme immunoassay. The sample may be a urine sample that contains a sum of cortisol, cortisone, alpha-tetrahydrocortiso, beta-tetrahydrocortisol and tetrahydrocortisone greater than or equal to 3.44 mg per mg of creatinine. According to a particular embodiment of the method, the sample is a plasma sample, and an amount of greater than or equal to 9 pg/mL AVP in the sample indicates that the subject is a suitable candidate for treatment with a V_{1B} antagonist. In this embodiment, the amount of AVP can be determined by radioimmunoassay. According to a further particular embodiment of the method, the sample is a plasma sample, and an amount of greater than or equal to 2.75 ng/mL of copeptin in the sample indicates that the subject is a suitable candidate for treatment with a V_{1B} antagonist. In this embodiment, the amount of copeptin can be determined by enzyme immunoassay. According to a further particular embodiment of the method, the sample is a urine sample, and the sum of cortisol, cortisone, alpha-tetrahydrocortiso, beta-tetrahydrocortisol and tetrahydrocortisone in the sample being greater than or equal to 3.44 mg per mg of creatinine indicates that the subject is a suitable candidate for treatment with a V_{1B} antagonist.

In the methods described herein the sample may be a nucleic acid containing sample, serum, plasma, blood, urine, or a saliva. The marker may be detected by an immunoassay. The marker may be detected by mass spectrometry.

In particular, the methods described above are used for subjects at risk of having or suspected to have a disorder characterized by HPA axis dysregulation. The disorder may be Cushing's syndrome, dementia, cognitive impairment, mood disorder, anxiety disorder, substance-related disorder, osteoporosis, arthritis, diabetes, dyslipidemia, obesity, hypertension, pain, glaucoma, or a combination thereof. The mood disorder may be depression. The depression may be major depressive disorder. The anxiety disorder post-traumatic stress disorder, generalized anxiety disorder, or panic disorder. The substance-related disorder may be alcohol dependence or abuse, or drug dependence or abuse. The dementia may be of the Alzheimer's type. The cognitive impairment may be mild cognitive impairment due to Alzheimer's disease.

In another aspect, the present invention is directed to a method for monitoring a subject's response to treatment with a V_{1B} antagonist. The method may comprise providing a biological sample from a subject and then detecting an HPA axis function marker in the sample. Alternatively, the method comprises detecting an HPA axis function marker in a biological sample obtained from the subject receiving treatment with the V_{1B} antagonist. If there is a greater than 25% change in the level of the marker as compared to a baseline, the V_{1B} antagonist is useful for treating the subject. The greater than 25% change may be an increase or decrease as compared to the baseline. The HPA axis function marker may be cortisol; cortisone; corticotrophin releasing hormone adrenocorticotrophin hormone (ACTH); hepatic metabolite of cortisol, hepatic metabolite of cortisone, or a combination thereof. The hepatic metabolite of cortisone may be tetrahydrocortisone, cortolone, cortolonic acid, or a combination thereof. The marker may be the sum of urine amounts of cortisol, cortisone, alpha-tetrahydrocortisol, beta-tetrahydrocortisol, and tetrahydrocortison. The marker may be the sum of urine amounts of alpha-tetrahydrocortisol, beta-tetrahydrocortisol, and tetrahydrocortisone. The sum of the urine amounts may be divided by the amount of creatinine in the same urine sample. The baseline may be cortisol at a level of between 3 pg/mL and 13 pg/mL. The baseline of cortisol may be determined using enzyme immunoassay. The baseline may indicate the level of the HPA axis function marker in a sample taken from the subject prior to beginning V_{1B} antagonist therapy. The ACTH may be plasma ACTH. According to certain embodiments of the invention, the method for monitoring a subject's response to treatment with a V_{1B} antagonist described herein is an in vitro method. The biological sample may be a nucleic acid containing sample, serum, plasma, blood, urine, or saliva. In particular, the method is used for subjects having a disorder characterized by HPA axis dysregulation. The disorder may be Cushing's syndrome, dementia, cognitive impairment, mood disorder, anxiety disorder, substance-related disorder, osteoporosis, arthritis, diabetes, dyslipidemia, obesity, hypertension, pain, glaucoma, or a combination thereof. The mood disorder may be depression. The depression may be major depressive disorder. The anxiety disorder may be post-traumatic stress disorder, generalized anxiety disorder or panic disorder. The substance-related disorder may be alcohol dependence or abuse, or drug dependence or abuse. The dementia may be of the Alzheimer's type. The cognitive impairment may be mild cognitive impairment due to Alzheimer's disease.

The V_{1B} antagonist may be of formula I:

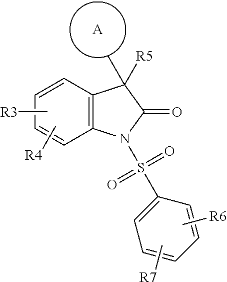

in which A is an aromatic heteromonocyclic ring, where the heterocycles are 5- or 6-membered rings and comprise up to 4 heteroatoms selected from the group consisting of N, O and S, where not more than one of the heteroatoms is an oxygen or sulfur atom, and A may be substituted by radicals R11, R12 and/or R13, where R11, R12 and R13 at each occurrence are selected independently of one another from the group consisting of hydrogen chlorine, bromine, iodine, fluorine, CN, CF₃, OCF₃, NO₂, OH, O—C₁-C₄-alkyl, O-phenyl, O—C₁-C₄-alkylen-phenyl, phenyl, C₁-C₆-alkyl, C₂-C₆-alkenyl, C₂-C₆-alkynyl, NH₂, NH(C₁-C₄-alkyl) and N(C₁-C₄-alkyl)₂, R3 and R4 are selected independently of one another from the group consisting of hydrogen, chlorine, bromine, iodine, fluorine, CN, CF₃, OCF₃, NO₂, OH, O—C₁-C₄-alkyl, O-phenyl, O—C₁-C₄-alkylen-phenyl, phenyl, C₁-C₆-alkyl, C₂-C₆-alkenyl, C₂-C₆-alkynyl, NH₂, NH(C₁-C₄-alkyl) and N(C₁-C₄-alkyl)₂, or R₃ and R₄ are connected to give —CH═CH—CH═CH—, —(CH₂)₄— or —(CH₂)₃—,

### R₅ is

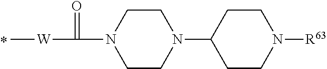

wherein W is selected from the group consisting of NR54, NR54-(C₁-C₄-alkylen) and a bond, R54 is independently selected from the group consisting of hydrogen, C₁-C₆-alkyl, C₂-C₆-alkenyl, C₂-C₆-alkynyl, phenyl and C₁-C₄-alkylen-phenyl, where the phenyl ring may be substituted by up to two radicals R59, R59 is independently selected from the group consisting of hydrogen, chlorine, bromine, iodine, fluorine, CN, CF₃, OCF₃, NO₂, OH, O—C₁-C₄-alkyl, C₁-C₆-alkyl, C₂-C₆-alkenyl, C₂-C₆-alkynyl, NH₂, NH(C₁-C₄-alkyl) and N(C₁-C₄-alkyl)₂, R63 is independently of one another from the group consisting of hydrogen, chlorine, bromine, iodine, fluorine, CN, CF₃, OCF₃, NO₂, OH, O—C₁-C₄-alkyl, O-phenyl, O—C₁-C₄-alkylen-phenyl, phenyl, C₁-C₆-alkyl, C₂-C₆-alkenyl, C₂-C₆-alkynyl, NH₂, NH(C₁-C₄-alkyl) and N(C₁-C₄-alkyl)₂, R6 and R7 are selected independently of one another from the group consisting of hydrogen, chlorine, bromine, iodine, fluorine, CN, CF₃, OCF₃, NO₂, OH, O—C₁-C₄-alkyl atoms, O-phenyl, O—C₁-C₄-alkylen-phenyl, phenyl, C₁-C₆-alkyl, C₂-C₆-alkenyl, C₂-C₆-alkynyl, NH₂, NH(C₁-C₄-alkyl) and N(C₁-C₄-alkyl)₂, and their tautomeric forms, enantiomeric and diastereomeric forms thereof.

A may be an aromatic heteromonocyclic system comprising 1 or 2 heteroatoms, wherein one of the 2 heteroatoms is nitrogen.

A may be pyrimidine, pyridine, pyridazine, pyrazine, thiazole, imidazole, thiophene- and furan.

The V_{1B} antagonist may be:

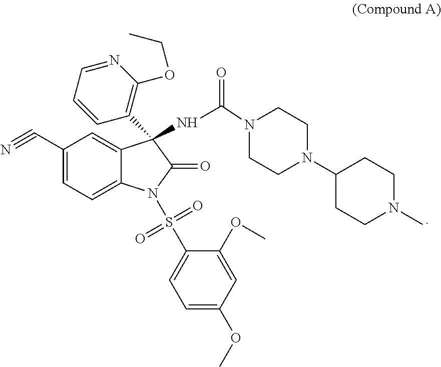 [SMILES: CCOc1ncccc1[C@]1(NC(=O)N2CCN(C3CCN(C)CC3)CC2)C(=O)N(S(=O)(=O)c2ccc(OC)cc2OC)c2ccc(C#N)cc21 ; "."]

The V_{1B} antagonist may be:

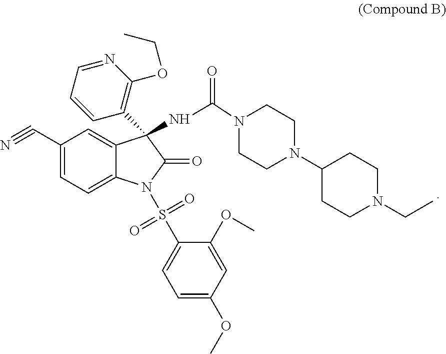 [SMILES: CCOc1ncccc1[C@]1(NC(=O)N2CCN(C3CCN(CC)CC3)CC2)C(=O)N(S(=O)(=O)c2ccc(OC)cc2OC)c2ccc(C#N)cc21 ; "."]

The herein described methods may be used to screen subjects for eligibility for a clinical trial. The methods may be used to stratify randomization of subjects for a clinical trial. The methods may be used to stratify analysis of a clinical trial.

In another aspect, the present invention is directed to a kit for stabilizing AVP in a plasma sample, wherein the kit comprises one or more collection tubes comprising one or more protease inhibitors. The kit may be used in the methods described herein.

In another aspect, the present invention is directed to a kit for assay of AVP in a blood-derived matrix, wherein the kit comprises a collection tube and instructions for stabilizing AVP at room temperature.

The present invention provides a V_{1B} antagonist as described herein for use in treating a subject identified as a suitable candidate by the method described herein. In particular, the subject to be treated is a subject at risk of having or a subject having a disorder characterized by HPA axis dysregulation, wherein a HPA axis function marker is present in a sample obtained from the subject. The present invention further provides a V_{1B} antagonist as described herein for use in treating a subject, wherein the treatment is continued if there is a greater than 25% change in the level of an HPA axis function marker in a biological sample obtained from the subject as compared to a baseline. The present invention also further provides a V_{1B} antagonist as described herein for use in treating a subject, wherein the treatment is discontinued if the change in the level of an HPA axis function marker in a biological sample obtained from the subject as compared to a baseline is 25% or less. Preferably, the V_{1B} antagonist is selected from ABT-436 and ABT-558.

### BRIEF DESCRIPTION OF THE DRAWINGS

FIG. 1 shows baseline AVP levels from clinical trial subjects who have major depressive disorder (MDD) as compared to AVP levels from health normal volunteers (HNV). Samples were drawn at approximately 8 am. The dashed line represents the 8.6 pg/mL cutoff value for high AVP defined using receiver-operator characteristic analysis with symptom change of ≦−0.53 points on the GDM subscale of MASQ as the dependent variable.

FIG. 2 shows data related to the interaction of AVP (high vs. normal) and treatment (Compound A (800 mg QD) vs. placebo), wherein the dependent variables are the Mood and Anxiety Symptom Questionnaire (MASQ) scale scores and Hamilton Depression Rating Scale (HAM-D) scores on Day 7 of study drug administration. High AVP was defined as ≧8.6 pg/mL, in a sample drawn at approximately 8 am or 2 pm on the day prior to initiation of study drug administration. Least squares means and standard errors are shown from analyses of covariance that included Day −1 (baseline) score as a covariate and factors for investigator, treatment, AVP class and treatment * AVP class interaction.

FIG. 3 shows data related to the interaction of AVP (high vs. normal) and treatment (Compound A vs. placebo), wherein the dependent variable is cortisol reactivity to a CRH challenge (maximum post-CRH serum cortisol divided by pre-CRH serum cortisol). High AVP was defined as ≧8.6 pg/mL, in a sample drawn at approximately 8 am or 2 pm on the day prior to initiation of study drug administration. The analysis of covariance model for Day 7 cortisol reactivity included Day −1 (baseline) cortisol reactivity as a covariate and factors for investigator, treatment, AVP class and treatment * AVP class interaction.

FIG. 4 shows a graph of the correlation between baseline copeptin and AVP levels from clinical trial subjects who have major depressive disorder (MDD). Samples were drawn at approximately 8 am, 2 pm and 10 pm prior to study drug initiation and after administration of the sixth dose of study drug. The black lines represent the 8.6 pg/mL cutoff values for high AVP, and the 2.8 ng/mL cutoff value for high copeptin, defined using receiver-operator characteristic analysis with symptom change of ≦−0.53 points on the GDM subscale of MASQ as the dependent variable. The dark gray line represents the best fit correlation of AVP and copeptin. The Spearman rank correlation coefficient is 0.56.

FIG. 5 shows a graph of the association between a panel of HPA- or depression-related variants and baseline AVP levels. The pharmacogenetic test result was considered positive if an individual had both rs7088418 AA and rs17169521 GG genotypes. AVP samples were drawn at approximately 2 pm. The grey diamonds represent group means and confidence intervals of the means.

FIG. 6 shows data related to the interaction of copeptin (high vs. normal) and treatment (Compound A vs. placebo), wherein the dependent variables are the Mood and Anxiety Symptom Questionnaire (MASQ) scale scores and Hamilton Depression Rating Scale (HAM-D) scores on Day 7 of study drug administration. High copeptin was defined as ≧2.8 ng/mL in a sample drawn at approximately 8 am or 2 pm on the day prior to initiation of study drug administration. Least squares means and standard errors are shown for analyses of covariance that included Day −1 (baseline) score as a covariate and factors for investigator, treatment, copeptin class and treatment * copeptin class interaction.

FIG. 7 shows data related to the interaction of a pharmacogenetic test result (positive vs. negative) and treatment (Compound A vs. placebo), wherein the dependent variables are MASQ scale scores and HAM-D scores on Day 7 of study drug administration. The pharmacogenetic test result was considered positive if an individual had both rs7088418 AA and rs17169521 GG genotypes. Least squares means and standard errors are shown from analyses of covariance that included Day −1 (baseline) score as a covariate and factors for investigator, treatment, pharmacogenetic test result and treatment * pharmacogenetic test result interaction.

FIG. 8 shows data related to the interaction of a pharmacogenetic test result (GG vs. A+(AG or AA)) and treatment (Compound A vs. placebo), wherein the dependent variables are the MASQ scale scores and HAM-D scores on Day 7 of study drug administration. The pharmacogenetic test result was based on rs 17100236. Least squares means and standard errors are shown from analyses of covariance that included Day −1 (baseline) score as a covariate and factors for investigator, treatment, pharmacogentic test result and treatment * pharmacogenetic test result interaction.

FIG. 9 shows data related to the interaction of urine glucocorticoid amount divided by urine creatinine amount (higher vs. lower) and treatment (Compound A vs. placebo), wherein the dependent variables are the Mood and Anxiety Symptom Questionnaire (MASQ) scale scores and Hamilton Depression Rating Scale (HAM-D) scores on Day 7 of study drug administration. Higher urine glucocorticoid amount was defined as ≧3.44 mg per mg creatinine, in a 24 hour collection on the day prior to initiation of study drug administration. Least squares means and standard errors are shown from analyses of covariance that included Day-1 (baseline) score as a covariate and factors for investigator, treatment, urine glucocorticoid class and treatment * urine glucocorticoid class interaction.

FIG. 10 shows data related to the interaction of urine glucocorticoid amount divided by urine creatinine amount prior to study drug administration (higher vs. lower) and treatment (Compound A vs. placebo), wherein the dependent variable is urine glucocorticoid amount divided by urine creatinine amount during study drug administration. Higher urine glucocortioids was defined as ≧3.44 mg per mg creatinine, in a 24-hour collection on the day prior to initiation of study drug administration. The analysis of covariance model for Day 6 to 7 urine glucocorticoids included Day −2 to −1 (baseline) urine glucocorticoids as a covariate and factors for investigator, treatment and treatment * baseline urine glucocorticoids interaction.

### DETAILED DESCRIPTION

The inventors have made the surprising discovery that there is an association between disorders related to HPA axis dysfunction and certain biological and genetic markers, as well as the predictive clinical value of these markers for response to a V_{1B} antagonist. The genetic identification, biochemical identification, or combination thereof, of one or more of these markers, or HPA axis function markers (“HPA markers”), in a sample from a subject may be useful in predicting whether the subject will respond to treatment with a V_{1B} antagonist, screening subject for eligibility for a clinical trial, stratifying randomization of subjects for a clinical trial, or stratifying analysis of a clinical trial.

The ability to target populations expected to show the highest clinical benefit, based on an HPA function marker profile, may improve the utilization of a drug for the benefit of a subject.

### (1) HPA AXIS SYSTEM BIOLOGY

The HPA axis system functions by the coordinated activity of hormone producing organs, which form a signaling system and ultimately result in the release of cortisol from the adrenal glands. See Chrousos and Gold (1992) J. Amer. Med. Assoc. 267, 1244-1252; and Kaltas and Chrousos (2007), Handbook of Psychophysiology, pp. 303-318, New York: Cambridge University Press. AVP produced by magnocellular neurons is carried to the posterior pituitary gland by neurosecretory granules. The AVP is then stored by the pituitary gland until it is released in the blood. Parvocellular neurons, in the paraventricular nucleus (PVN) of the hypothalamus, secrete corticotrophin-releasing hormone (CRH) and AVP into portal circulation to act at the anterior pituitary. While CRH and AVP stimulate corticotrophic cells in the pituitary to release adrenocorticotropic hormone (ACTH), AVP acts specifically on the pituitary gland receptor, V_{1B}. The ACTH travels via blood to the adrenal glands, where it triggers the release of corticosteroids, including cortisol and dehydroepiandrosterone (DHEA).

Cortisol reduces CRH in the PVN of the HPA axis system. The HPA axis system is an auto-regulating system that can decrease its activity via a negative feedback loop. The reduction of CRH results in the decreased cortisol and DHEA by the adrenal glands. This auto-regulation protects various tissues from extended exposure to high levels of cortisol as hypercortisolism can lead to a variety of harmful immunological, metabolic, and psychological side effects. See Review of Guerry and Hastings, Clin. Child. Fam. Psychol. Rev. (2011)14:135-160.

The HPA axis is active over the circadian cycle of day and night. Cortisol levels typically follow a predictable pattern over this circadian cycle. See Lovallo and Thomas (2000) Handbook of psychophysiology, pp. 342-367, Cambridge, UK.: Cambridge University Press. Cortisol levels may be high by the end of the sleeping period and continue to increase until they peak 30 to 40 minutes after awakening. This is known as the “cortisol awakening response” (CAR). Accordingly, during the awake/day hours, cortisol levels may be depicted by a negative slope; cortisol levels then increase again during sleep.

The diurnal rhythm may constitute a “baseline” or “basal” or “tonic” HPA activity, which may represent the amount of cortisol that would be expected to be circulating in the blood at a given time of day. HPA axis functioning is at the center of the body's response to acute “stressors” by changing its level of activity. For example, the HPA axis triggers an increase in circulating cortisol levels when a challenging or threatening event occurs. After the HPA axis activity is induced and increased, it typically will take about 20 minutes to reach peak levels circulating cortisol. See Gunnar and Talge (2006) Developmental psychophysiology: Theory, systems, and methods, pp. 343-366; New York: Cambridge University Press; and Kudielka et al., (2004) Psychoneuroendocrinology, 29, 983-992. The intensity and duration of the stressful event, or stressor, may impact the length of time it will take for the circulating cortisol to return to the baseline expected for the time of day. The normal development of HPA axis activity results in higher basal cortisol levels and stronger HPA axis responses to stressful events in adolescence, when the frequency of stressors and the prevalence of depression also increase. See Review of Guerry and Hastings, Clin. Child. Fam. Psychol. Rev. (2011)14:135-160.

### 2. DEFINITIONS

The terminology used herein is for the purpose of describing particular embodiments only and is not intended to be limiting. As used in the specification and the appended claims, the singular forms “a,” “and” and “the” include plural references unless the context clearly dictates otherwise.

a. About

“About” as used herein may refer to approximately a +/−10% variation from the stated value. It is to be understood that such a variation is always included in any given value provided herein, whether or not specific reference is made to it.

b. Identical or Identity

“Identical” or “identity,” as used herein in the context of two or more polypeptide or polynucleotide sequences, can mean that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. The residues of the single sequence are included in the denominator, but not the numerator of the calculation, in cases where the two sequences are of different lengths or the alignment produces one or more staggered ends and the specified region of comparison includes only a single sequence.

c. Isolated Polynucleotide

“Isolated polynucleotide” as used herein may mean a polynucleotide (e.g. of genomic, cDNA, or synthetic origin, or a combination thereof) that, by virtue of its origin, the isolated polynucleotide is not associated with all or a portion of a polynucleotide with which the “isolated polynucleotide” is found in nature; is operably linked to a polynucleotide that it is not linked to in nature; or does not occur in nature as part of a larger sequence.

d. Label and Detectable Label

“Label” and “detectable label” as used herein refer to a moiety attached to an antibody or an analyte to render the reaction between the antibody and the analyte detectable, and the antibody or analyte so labeled is referred to as “detectably labeled.” A label can produce a signal that is detectable by visual or instrumental means. Various labels include signal-producing substances, such as chromogens, fluorescent compounds, chemiluminescent compounds, radioactive compounds, and the like. Representative examples of labels include moieties that produce light, e.g., acridinium compounds, and moieties that produce fluorescence, e.g., fluorescein. Other labels are described herein. In this regard, the moiety, itself, may not be detectable but may become detectable upon reaction with yet another moiety. Use of the term “detectably labeled” is intended to encompass such labeling.

Any suitable detectable label as is known in the art can be used. For example, the detectable label can be a radioactive label (such as ³H, ¹²⁵I, ³⁵S, ¹⁴C, ³²P, and ³³P), an enzymatic label (such as horseradish peroxidase, alkaline peroxidase, glucose 6-phosphate dehydrogenase, and the like), a chemiluminescent label (such as acridinium esters, thioesters, or sulfonamides; luminol, isoluminol, phenanthridinium esters, and the like), a fluorescent label (such as fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2^{nd} ed., Springer Verlag, N.Y. (1997), and in Haugland, Handbook of Fluorescent Probes and Research Chemicals (1996), which is a combined handbook and catalogue published by Molecular Probes, Inc., Eugene, Oreg. A fluorescent label can be used in FPIA (see, e.g., U.S. Pat. Nos. 5,593,896, 5,573,904, 5,496,925, 5,359,093, and 5,352,803, which are hereby incorporated by reference in their entireties). An acridinium compound can be used as a detectable label in a homogeneous chemiluminescent assay (see, e.g., Adamczyk et al., Bioorg. Med. Chem. Lett. 16: 1324-1328 (2006); Adamczyk et al., Bioorg. Med. Chem. Lett. 4: 2313-2317 (2004); Adamczyk et al., Biorg. Med. Chem. Lett. 14: 3917-3921 (2004); and Adamczyk et al., Org. Lett. 5: 3779-3782 (2003)).

In one aspect, the acridinium compound is an acridinium-9-carboxamide. Methods for preparing acridinium 9-carboxamides are described in Mattingly, J. Biolumin. Chemilumin. 6: 107-114 (1991); Adamczyk et al., J. Org. Chem. 63: 5636-5639 (1998); Adamczyk et al., Tetrahedron 55: 10899-10914 (1999); Adamczyk et al., Org. Lett. 1: 779-781 (1999); Adamczyk et al., Bioconjugate Chem. 11: 714-724 (2000); Mattingly et al., In Luminescence Biotechnology Instruments and Applications; Dyke, K. V. Ed.; CRC Press: Boca Raton, pp. 77-105 (2002); Adamczyk et al., Org. Lett. 5: 3779-3782 (2003); and U.S. Pat. Nos. 5,468,646, 5,543,524 and 5,783,699 (each of which is incorporated herein by reference in its entirety for its teachings regarding same).

Another example of an acridinium compound is an acridinium-9-carboxylate aryl ester. An example of an acridinium-9-carboxylate aryl ester of formula II is 10-methyl-9-(phenoxycarbonyl)acridinium fluorosulfonate (available from Cayman Chemical, Ann Arbor, Mich.). Methods for preparing acridinium 9-carboxylate aryl esters are described in McCapra et al., Photochem. Photobiol. 4: 1111-21 (1965); Razavi et al., Luminescence 15: 245-249 (2000); Razavi et al., Luminescence 15: 239-244 (2000); and U.S. Pat. No. 5,241,070 (each of which is incorporated herein by reference in its entirety for its teachings regarding same). Such acridinium-9-carboxylate aryl esters are efficient chemiluminescent indicators for hydrogen peroxide produced in the oxidation of an analyte by at least one oxidase in terms of the intensity of the signal or the rapidity of the signal. The course of the chemiluminescent emission for the acridinium-9-carboxylate aryl ester is completed rapidly, i.e., in under 1 second, while the acridinium-9-carboxamide chemiluminescent emission extends over 2 seconds. Acridinium-9-carboxylate aryl ester, however, loses its chemiluminescent properties in the presence of protein. Therefore, its use requires the absence of protein during signal generation and detection. Methods for separating or removing proteins in the sample are well-known to those skilled in the art and include, but are not limited to, ultrafiltration, extraction, precipitation, dialysis, chromatography, or digestion (see, e.g., Wells, High Throughput Bioanalytical Sample Preparation. Methods and Automation Strategies, Elsevier (2003)). The amount of protein removed or separated from the test sample can be about 40%, about 45%, about 50%, about 55%, about 60%, about 65%, about 70%, about 75%, about 80%, about 85%, about 90%, or about 95%. Further details regarding acridinium-9-carboxylate aryl ester and its use are set forth in U.S. patent application Ser. No. 11/697,835, filed Apr. 9, 2007. Acridinium-9-carboxylate aryl esters can be dissolved in any suitable solvent, such as degassed anhydrous N,N-dimethylformamide (DMF) or aqueous sodium cholate.

e. Normal Subject Sample

“Normal subject sample” as used herein may refer to a sample from a subject who is free from a disease or disorder related to HPA axis dysfunction. The normal subject sample may be a control. The normal subject sample may be from a geriatric or pediatric subject.

f. Predetermined Cutoff and Predetermined Level

“Predetermined cutoff” and “predetermined level” refer generally to an assay cutoff value that is used to assess diagnostic/prognostic/therapeutic efficacy results by comparing the assay results against the predetermined cutoff/level, where the predetermined cutoff/level already has been linked or associated with various clinical parameters (e.g., severity of disease, progression/nonprogression/improvement, etc.). The present disclosure provides exemplary predetermined levels. However, it is well-known that cutoff values may vary depending on the nature of the immunoassay (e.g., antibodies employed, etc.). It further is well within the ordinary skill of one in the art to adapt the disclosure herein for other immunoassays to obtain immunoassay-specific cutoff values for those other immunoassays based on this disclosure. Whereas the precise value of the predetermined cutoff/level may vary between assays, the correlations as described herein should be generally applicable.

g. Quality Control Reagents

“Quality control reagents” in the context of immunoassays and kits described herein, include, but are not limited to, calibrators, controls, and sensitivity panels. A “calibrator” or “standard” typically is used (e.g., one or more, such as a plurality) in order to establish calibration (standard) curves for interpolation of the concentration of an analyte, such as an antibody or an analyte. Alternatively, a single calibrator, which is near a predetermined positive/negative cutoff, can be used. Multiple calibrators (i.e., more than one calibrator or a varying amount of calibrator(s)) can be used in conjunction so as to comprise a “sensitivity panel.”

h. Series of Calibrating Compositions

“Series of calibrating compositions” refers to a plurality of compositions comprising a known concentration of an HPA marker, wherein each of the compositions differs from the other compositions in the series by the concentration of the HPA marker.

i. Solid Phase

“Solid phase” refers to any material that is insoluble, or can be made insoluble by a subsequent reaction. The solid phase can be chosen for its intrinsic ability to attract and immobilize a capture agent. Alternatively, the solid phase can have affixed thereto a linking agent that has the ability to attract and immobilize the capture agent. The linking agent can, for example, include a charged substance that is oppositely charged with respect to the capture agent itself or to a charged substance conjugated to the capture agent. In general, the linking agent can be any binding partner (preferably specific) that is immobilized on (attached to) the solid phase and that has the ability to immobilize the capture agent through a binding reaction. The linking agent enables the indirect binding of the capture agent to a solid phase material before the performance of the assay or during the performance of the assay. The solid phase can, for example, be plastic, derivatized plastic, magnetic or non-magnetic metal, glass or silicon, including, for example, a test tube, microtiter well, sheet, bead, microparticle, chip, and other configurations known to those of ordinary skill in the art.

j. Specific Binding

“Specific binding” or “specifically binding” as used herein may refer to the interaction of an antibody, a protein, or a peptide with a second chemical species, wherein the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

k. Specific Binding Partner

“Specific binding partner” is a member of a specific binding pair. A specific binding pair comprises two different molecules, which specifically bind to each other through chemical or physical means. Therefore, in addition to antigen and antibody specific binding pairs of common immunoassays, other specific binding pairs can include biotin and avidin (or streptavidin), carbohydrates and lectins, complementary nucleotide sequences, effector and receptor molecules, cofactors and enzymes, enzymes and enzyme inhibitors, and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes and fragments thereof, whether isolated or recombinantly produced.

l. Stringent Conditions

“Stringent conditions” is used herein to describe hybridization to filter-bound DNA in 6× sodium chloride/sodium citrate (SSC) at about 45° C. followed by one or more washes in 0.2×SSC/0.1% SDS at about 50-65° C. The term “under highly stringent conditions,” refers to hybridization to filter-bound nucleic acid in 6×SSC at about 45° C. followed by one or more washes in 0.1×SSC/0.2% SDS at about 68° C., or under other related conditions. See, for example, Ausubel, F. M. et al., eds., 1989, Current Protocols in Molecular Biology, Vol. I, Green Publishing Associates, Inc. and John Wiley & Sons, Inc., New York at pages 6.3.1-6.3.6 and 2.10.3.

m. Treat, Treating or Treatment

“Treat”, “treating” or “treatment” are each used interchangeably herein to describe reversing, alleviating, or inhibiting the progress of a disorder/disease, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of an antibody or pharmaceutical composition of the present invention to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

n. Variant

“Variant” is used herein to describe a peptide or polypeptide that differs in amino acid sequence by the insertion, deletion, or conservative substitution of amino acids, but retains at least one biological activity. Representative examples of “biological activity” include the ability to be bound by a specific antibody or to promote an immune response. Variant is also used herein to describe a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. “Variant” also can be used to refer to an antigenically reactive fragment of an anti-HPA marker antibody that differs from the corresponding fragment of anti-HPA marker antibody in amino acid sequence but is still antigenically reactive and can compete with the corresponding fragment of anti-HPA marker antibody for binding with an HPA marker. “Variant” also can be used to describe a polypeptide or a fragment thereof that has been differentially processed, such as by proteolysis, phosphorylation, or other post-translational modification, yet retains its antigen reactivity.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 3. METHOD OF IDENTIFYING HPA AXIS FUNCTION MARKERS

Provided herein is a method for detecting an HPA axis function marker in a biological sample. The sample may be from a subject afflicted with a disorder characterized by HPA axis dysregulation. The HPA marker may be a genomic, non-genomic, or a combination thereof. Depending on the type of HPA marker, the method of detection can be an immunoassay or genotyping, for example. According to certain embodiments of the invention, the method is an in vitro method.

a. Method for Determining Whether Subject is Suitable for V_{1B} Antagonist Treatment

The method for detecting an HPA axis marker may be applied to a method for determining whether a subject is a suitable candidate for treatment with a V_{1B} antagonist wherein the subject has a disorder associated with HPA axis dysfunction. The method comprises providing a biological sample from the subject and detecting an HPA axis function marker, wherein the presence of an HPA marker in the subject's sample or particular level of the HPA marker over a normal subject sample may indicate that the subject is suitable for treatment with the V_{1B} antagonist. Alternatively, the method comprises detecting an HPA axis function marker in a biological sample obtained from the subject, wherein the presence of an HPA marker in the subject's sample or particular level of the HPA marker over a normal subject sample indicates that the subject is suitable for treatment with the V_{1B} antagonist. The knowledge of the presence of a particular marker, or level thereof, associated with HPA dysfunction will allow one to customize the prevention or treatment in accordance with the subject's HPA axis function marker profile. For example, a subject identified as being a suitable candidate pursuant to the methods described herein can be administered (namely, treated with) at least one V_{1B} antagonist. The HPA marker may be genomic. The HPA marker may be non-genomic, such as a hormone, a protein, a fragment thereof, or any combination thereof. The method for identifying an HPA marker may also be used for screening subjects for eligibility for a clinical trial, to stratify randomization of subjects for a clinical trial, to stratify analysis of a clinical trial, or a combination thereof

b. Method for Monitoring a Subject's Response to V_{1B} Antagonist Treatment

Provided herein is a method for identifying one or more “monitoring” HPA markers in a sample from the subject, which may be applied to a method for monitoring the subject's response to V_{1B} antagonist treatment.

The monitoring HPA axis marker may be detected in a biological sample from the subject. A greater than 25% change in the level of the marker as compared to a baseline may indicate that the subject is responding to the V_{1B} antagonist treatment and that the V_{1B} antagonist may be useful for treating the subject. This change may be an increase change or decrease change as compared to the baseline. The subject may have a disorder associated with HPA axis dysfunction. If the subject is determined to be responding to the V_{1B} antagonist treatment pursuant to the methods described herein, then treatment with the V_{1B} antagonist treatment may be continued. If the subject is determined not to be responding to the V_{1B} antagonist treatment pursuant to the methods described herein, then treatment with the V_{1B} antagonist treatment may be discontinued.

c. HPA Marker—to be Used in Method of Identifying HPA Axis Function Markers

The HPA marker of the methods described above may be a genomic HPA marker, non-genomic HPA marker, or a combination thereof. The HPA axis marker may be detected in a sample from the subject. The presence of the genomic marker may indicate that the subject is a suitable candidate for V_{1B} antagonist treatment. If the method detects the presence of a non-genomic HPA axis marker, the level of the HPA marker may be measured. If the level is above a particular percentile of the distribution of the marker in a normal subject sample, then the subject may be a suitable candidate for V_{1B} antagonist treatment.

(a) Genomic HPA Marker

The genetic marker may be a deletion, substitution, insertion, or a polymorphism. The genomic HPA marker may be a polymorphism in any of the LHPP, AKRID1, or NR3C1 genes, or fragments thereof. The HPA marker may be any combination of genetic markers. The polymorphism may be a single nucleotide polymorphism. Within a population, a marker may be assigned a minor allele frequency. There may be variations between subject populations. A marker that is common in one geographical or ancestral group may be rarer in another. The marker may be overrepresented in a group of subjects that have a disorder associated with HPA axis dysregulation. Subjects that have a disorder associated with HPA axis dysregulation may be divided into groups on the basis of age, sex, ancestry, or a combination thereof

(a) LHPP

The gene, LHPP, is a Major Depressive Disorder (MDD)-linked gene that encodes an enzyme known as phospholysine phosphohistidine inorganic pyrophosphate phosphatase, and the term “LHPP” refers to the corresponding messenger RNA, and DNA sequences that functionally regulate expression thereof LHPP was originally purified from swine brain in 1957 (See, Seal et al., J Biol Chem 228:193-9 (1957)), and subsequently has been purified from several additional mammalian sources (See, Felix et al., J Biochem 147:111-8 (1975); Yoshida et al., Cancer Research 42:3256-31 (1982); Hachimori et al., J Biochem 93:257-64 (1983); Smirnova et al., Arch Biochem Biophys 287:135-40 (1991); Hiraishi et al., Arch Biochem Biophys 341:153-9 (1997)). The enzyme has been characterized in vitro as efficiently catalyzing the hydrolysis of P—N bonds in phosphohistidine and phospholysine, and less efficiently catalyzing the hydrolysis of P—N or P—O bonds in imidodiphosphate and pyrophosphate, respectively. LHPP may be a protein histidine or lysine phosphoamidase, i.e., an enzyme that modifies the N-linked phosphorylation state of other proteins. The human LHPP has been cloned. Functional human LHPP enzyme has been purified following heterologous expression in E. coli (See, Yokoi et al., J Biochem 133:607-14 (2003)). LHPP has been genetically linked and associated with major depressive disorder. See Neff et al., Mol. Psychiatry, 14:621-630 (2009).

The genetic marker may be a nucleotide sequence that comprises SEQ ID NO:1 (LHPP rs7088418).

(b) AKR1D1

The gene, AKR1D1, encodes a protein that converts cortisol and cortisone to tetrahydro metabolites in the liver. More specifically, the enzyme encoded by this gene is responsible for the catalysis of the 5-beta-reduction of bile acid intermediates and steroid hormones carrying a delta(4)-3-one structure. Deficiency of this enzyme may contribute to hepatic dysfunction. Three transcript variants encoding different isoforms have been found for this gene.

The genetic marker may be SEQ ID NO:2 (AKRID1 rs17169521).

(c) NR3C1

The gene, NR3C1, encodes glucocorticoid receptor, which can function both as a transcription factor that binds to glucocorticoid response elements in the promoters of glucocorticoid responsive genes to activate their transcription, and as a regulator of other transcription factors. This receptor is typically found in the cytoplasm, but upon ligand binding, is transported into the nucleus. It is involved in inflammatory responses, cellular proliferation, and differentiation in target tissues. Mutations in this gene are associated with generalized glucocorticoid resistance.

The genetic marker may be a nucleotide sequence that comprises an NR3C1 genotype. The NR3C1 genotype may be a nucleic acid that comprises SEQ ID NO:3 (rs10482672), SEQ ID NO:4 (rs17100236), or a combination thereof.

(b) Non-Genomic HPA Marker

The HPA marker may be a non-genomic HPA marker, such as a protein or peptide. The peptide may be a peptide hormone, such as arginine vasopressin (AVP), or a fragment of preprovasopressin, such as copeptin or neurophysin II. The non-genomic HPA marker may be cortisol, cortisone, corticotrophin releasing hormone, adrenocorticotrophin hormone, or a combination thereof. The sum of cortisol, cortisone, and hepatic metabolites thereof from one or more samples, such as urine, may be ascertained via the methods described herein.

(a) AVP

AVP may have the following amino acid sequence: Cys-Tyr-Phe-Gln-Asn-Cys-Pro-Arg-Gly-NH₂ (SEQ ID NO:5). The subject may have baseline AVP levels above the 60^{th} percentile, the 70^{th} percentile, the 80^{th} percentile, the 81^{st}, 82^{nd}, 83^{rd}, 84^{th}, 85^{th}, 86^{th}, 87^{th}, 88^{th}, 89^{th}, 90^{th}, 91^{st}, 92^{nd}, 93^{rd}, 94^{th}, 95^{th}, 96^{th}, 97^{th}, 98^{th}, or 99^{th} percentile of a distribution of AVP in a sample from a normal subject or of AVP in a lab reference. The subject with a disorder related to HPA axis function may have a baseline blood, serum, or plasma AVP level of greater than a concentration between 2 pg/mL and 15 pg/mL, greater than a concentration between 5 pg/mL and 15 pg/mL, greater than a concentration between 5 pg/mL and 14 pg/mL, greater than a concentration between 5 pg/mL and 13 pg/mL, greater than a concentration between 6 pg/mL and 12 pg/mL, greater than a concentration between 7 pg/mL and 11 pg/mL, or greater than a concentration between 8 pg/mL and 10 pg/mL. The subject with a disorder related to HPA axis function may have a baseline blood, serum, or plasma AVP level of greater than 2 pg/mL, 3 pg/mL, 4 pg/mL, 5 pg/mL, 6 pg/mL, 7 pg/mL, greater than 8 pg/mL, greater than 8.1 pg/mL, greater than 8.2 pg/mL, greater than 8.3 pg/mL, greater than 8.4 pg/mL, greater than 8.5 pg/mL, greater than 8.6 pg/mL, greater than 8.7 pg/mL, greater than 8.8, pg/mL, greater than 8.9 pg/mL greater than 9 pg/mL, greater than 9.5 pg/mL, greater than 10 pg/mL, or greater than 11 pg/mL. The subject with a disorder related to HPA axis function may have a baseline blood, serum, or plasma AVP level of 2 pg/mL, 3 pg/mL, 4 pg/mL, 5 pg/mL, 6 pg/mL, 7 pg/mL, 8 pg/mL, 8.1 pg/mL, 8.2 pg/mL, 8.3 pg/mL, 8.4 pg/mL, 8.5 pg/mL, 8.6 pg/mL, 8.7 pg/mL, 8.8, pg/mL, 8.9 pg/mL, 9 pg/mL, 9.5 pg/mL, 10 pg/mL, or 11 pg/mL. The level may be determined against a lab reference. The blood, serum, or plasma AVP level may be measured at anytime; for example, in the morning, afternoon, or evening of any given day.

The subject may have a baseline blood, serum, or plasma AVP level of greater than 2 pg/mL, 3 pg/mL, 4 pg/mL, 5 pg/mL, 6 pg/mL, 7 pg/mL, 8 pg/mL, 8.1 pg/mL, 8.2 pg/mL, 8.3 pg/mL, 8.4 pg/mL, 8.5 pg/mL, 8.6 pg/mL, 8.7 pg/mL, 8.8, pg/mL, 8.9 pg/mL, 9 pg/mL, 9.1 pg/mL, 9.2 pg/mL, 9.3 pg/mL, 9.4 pg/mL, 9.5 pg/mL, 9.6 pg/mL, 9.7 pg/mL, 9.8, pg/mL, 9.9 pg/mL 10 pg/mL, 11 pg/mL, 12 pg/mL, 13 pg/mL, 13.1 pg/mL, 13.2 pg/mL, 13.3 pg/mL, 13.4 pg/mL, 13.5 pg/mL, 13.6 pg/mL, 13.7 pg/mL, 13.8, pg/mL, 13.9 pg/mL, 14 pg/mL, 14.1 pg/mL, 14.2 pg/mL, 14.3 pg/mL, 14.4 pg/mL, 14.5 pg/mL, 14.6 pg/mL, 14.7 pg/mL, 14.8, pg/mL, 14.9 pg/mL, or 15 pg/mL as measured using a commercially available immunoassay service or kit. The subject may have a baseline blood, serum, or plasma AVP level of 2 pg/mL, 3 pg/mL, 4 pg/mL, 5 pg/mL, 6 pg/mL, 7 pg/mL, 8 pg/mL, 8.1 pg/mL, 8.2 pg/mL, 8.3 pg/mL, 8.4 pg/mL, 8.5 pg/mL, 8.6 pg/mL, 8.7 pg/mL, 8.8, pg/mL, 8.9 pg/mL, 9 pg/mL, 9.1 pg/mL, 9.2 pg/mL, 9.3 pg/mL, 9.4 pg/mL, 9.5 pg/mL, 9.6 pg/mL, 9.7 pg/mL, 9.8, pg/mL, 9.9 pg/mL 10 pg/mL, 11 pg/mL, 12 pg/mL, 13 pg/mL, 13.1 pg/mL, 13.2 pg/mL, 13.3 pg/mL, 13.4 pg/mL, 13.5 pg/mL, 13.6 pg/mL, 13.7 pg/mL, 13.8, pg/mL, 13.9 pg/mL, 14 pg/mL, 14.1 pg/mL, 14.2 pg/mL, 14.3 pg/mL, 14.4 pg/mL, 14.5 pg/mL, 14.6 pg/mL, 14.7 pg/mL, 14.8, pg/mL, 14.9 pg/mL, or 15 pg/mL as measured using a commercially available immunoassay service or kit. For example, the AVP level may be measured by immunoassay in a laboratory of a commercial provider of such services. One such provider is Quest Diagnostics, with corporate headquarters at 3 Giralda Farms, Madison, N.J. 07940, United States of America. Another provider may be Thermo Fisher Scientific, Inc. Depending on the provider of the service and the kind of immunoassay performed, the lab reference range or standard may differ from that of another provider or immunoassay.

Practical limitations may exist with respect to assaying for AVP. For example, unless frozen, AVP is unstable ex vivo in plasma. The instability may be due to a reduction of disulfide bonds, which help to maintain the active conformation of AVP. Testing may be available through a limited number of reference labs, which typically have relatively long turnaround times for results. In addition, assay standard supplies are unreliable. These potential limitations may be overcome by assaying for one or more surrogate markers as described herein, such as, one or more genetic markers, protein markers, or other hormones.

(b) Copeptin

As described above, the preanalytic instability of AVP has limited previous tests related to this hormone, but the stable glycopeptide copeptin may serve as a surrogate because it is co-secreted with AVP. Copeptin, a 39-amino acid glycopeptide that comprises the C-terminal part of the AVP precursor was found to be a stable and sensitive surrogate marker for AVP release. Serum copeptin is glycosylated and contains a sugar moiety.

The subject may have baseline copeptin levels above the 60^{th} percentile, the 70^{th} percentile, 80^{th} percentile, the 81^{st}, 82^{nd}, 83^{rd}, 84^{th}, 85^{th}, 86^{th}, 87^{th}, 88^{th}, 89th, 90^{th}, 91^{st}, 92^{nd}, 93^{rd}, 94^{th}, 95^{th}, 96^{th}, 97^{th}, 98^{th}, or 99^{th} percentile of copeptin in a sample from a normal subject or of copeptin in a lab reference. The subject with a disorder related to HPA axis function may have a baseline blood, serum, or plasma copeptin level of greater than a concentration between 1.0 ng/mL and 8.0 ng/mL, between 2 ng/mL and 7 ng/mL, between 3 ng/mL and 6 ng/mL, between 3 ng/mL and 8 ng/mL, 1.5 ng/mL and 6 ng/mL, between 2 ng/mL and 5 ng/mL, or between 3 ng/mL and 5 ng/mL. The subject with a disorder related to HPA axis function may have a baseline blood, serum, or plasma copeptin level of greater than 1.5 ng/mL, greater than 2 ng/mL, greater than 2.5 ng/mL, greater than 2.75 ng/mL, greater than 2.8 ng/mL, greater than 3 ng/mL, greater than 4 ng/mL, greater than 5 ng/mL, greater than 6 ng/mL, greater than 7 ng/mL, or greater than 7.5 ng/mL. The subject with a disorder related to HPA axis function may have a baseline blood, serum, or plasma copeptin level that is 1.5 ng/mL, 2 ng/mL, 2.1 ng/mL, 2.2 ng/mL, 2.3 ng/mL, 2.4 ng/mL, 2.5 ng/mL, 2.6 ng/mL, 2.7 ng/mL, 2.8 ng/mL, 2.9 ng/mL, 3 ng/mL, 4 ng/mL, 5 ng/mL, 6 ng/mL, 7 ng/mL, or 7.5 ng/mL. The level may be determined against a lab reference. The blood, serum, or plasma copeptin level may be measured at anytime; for example, in the morning, afternoon, or evening of any given day.

The subject may have a baseline blood, serum, or plasma copeptin level of greater than 2.0 ng/mL, 2.25 ng/mL, 2.50 ng/mL, 2.75 ng/mL, or 3.0 ng/mL as measured using a commercially available immunoassay service or kit. The subject may have a baseline blood, serum, or plasma copeptin level that is 1.5 ng/mL, 2 ng/mL, 2.1 ng/mL, 2.2 ng/mL, 2.3 ng/mL, 2.4 ng/mL, 2.5 ng/mL, 2.6 ng/mL, 2.7 ng/mL, 2.8 ng/mL, 2.9 ng/mL, 3 ng/mL, 4 ng/mL, 5 ng/mL, 6 ng/mL, 7 ng/mL, or 7.5 ng/mL as measured using a commercially available immunoassay service or kit. For example, the copeptin level may be measured by immunoassay in a laboratory of a commercial provider of such services. One such provider is Quest Diagnostics, with corporate headquarters at 3 Giralda Farms, Madison, N.J. 07940, United States of America. One such commercially available assay is the B•R•A•H•M•S* Copeptin Immunoassay by Thermo Fisher Scientific, Inc. Depending on the provider of the service and the kind of immunoassay performed, the lab reference range or standard may differ from that of another provider or immunoassay. The level of blood, serum, or plasma copeptin may be expressed in pM.

Cortisol

As described above, cortisol production is a result of the intricate coordination of events in the HPA axis. More specifically, cortisol is one of the steroid hormones produced by the adrenal gland in the zona fasciculata. A cascade of signaling occurs for cortisol to be released from the adrenal gland. Corticotropin-releasing hormone and AVP released from the hypothalamus stimulates corticotrophs in the anterior pituitary to release ACTH, which relays the signal to the adrenal cortex. Here, the zona fasciculata and zona reticularis, in response to ACTH, secrete glucocorticoids, in particular cortisol. The cortisol may be a hepatic metabolite of cortisol. The hepatic metabolite of cortisol may be alpha-tetrahydrocortisol, beta-tetrahydrocortisol, alpha-cortol, beta-cortol, alpha-cortolic acid, beta-cortolic acid, or a combination thereof.

The subject may have baseline cortisol levels above the 60^{th} percentile, the 70^{th} percentile, the 80^{th} percentile, the 81^{st}, 82^{nd} 83^{rd}, 84^{th}, 85^{th}, 86^{th}, 87^{th}, 88^{th}, 89^{th}, 90^{th}, 91^{st}, 92^{nd}, 93^{rd}, 94^{th}, 95^{th}, 96^{th}, 97^{th}, 98^{th}, or 99^{th} percentile of cortisol in a sample from a normal subject or of cortisol in a lab reference. The subject with a disorder related to HPA axis function may have a baseline saliva, urine, blood, serum, or plasma cortisol level of greater than a concentration between 0.5 pg/mL and 6 pg/mL, between 1 pg/mL and 5 pg/mL, or between 2 pg/mL and 4 pg/mL. The subject with a disorder related to HPA axis function may have a baseline blood, urine, serum, saliva, or plasma cortisol level of greater than 0.5 pg/mL, greater than 2 pg/mL, greater than 2.5 pg/mL, greater than 2.75 pg/mL, greater than 3 pg/mL, greater than 3.5 pg/mL, greater than 4.0 pg/mL, greater than 4.5 pg/mL, greater than 4.75 pg/mL, greater than 5 pg/mL, greater than 5.5 pg/mL, or greater than 6 pg/mL. The level may be determined against a lab reference. The blood, urine, serum, saliva, or plasma cortisol level may be measured at anytime; for example, in the morning, afternoon, or evening of any given day. For example, urine cortisol may be measured at a single time. Urine cortisol may be measured in a 24-hour collection or overnight collection. The overnight collection may be a 5 hour, 6 hour, 7 hour, 8 hour, 9 hour, 10 hour, 11 hour, 12 hour or 13 hour collection, for example. Urine cortisol may be normalized by urine creatinine, which may be useful for a single time measurement or overnight collection. The cortisol level in blood may be measured as total cortisol. Free cortisol, or cortisol that is not bound to protein, may be estimated or calculated. The cortisol level in urine or saliva, for example, may be free cortisol, i.e. not bound to protein.

The cortisol level may be measured by mass spectrometry or immunoassay in a laboratory of a commercial provider of such services. One such provider is Quest Diagnostics, with corporate headquarters at 3 Giralda Farms, Madison, N.J. 07940, United States of America. Another provider may be a bioanalytical lab such as Pharmaceutical Product Development, Inc., or “PPD.” Another provider may be Thermo Fisher Scientific, Inc, or Abbott Laboratories. Commercially available assay kits may be available from Abbott Laboratories. Depending on the provider of the service and the kind of immunoassay performed, the lab reference range or standard may differ from that of another provider or immunoassay.

(c) Cortisone

Cortisone is one of several end-products of a process called steroidogenesis. This process starts with the synthesis of cholesterol, which then proceeds through a series of modifications in the adrenal gland (suprarenal) to become any one of many steroid hormones. One end-product of this pathway is cortisol, as described above. In peripheral tissues, cortisol is converted to cortisone by the enzyme 11-beta-steroid dehydrogenase. Cortisone is activated through hydrogenation of the 11-keto-group, and cortisol is, thus, sometimes referred to as hydrocortisone.

The subject may have baseline cortisone levels above the 60^{th} percentile, the 70^{th} percentile, the 80^{th} percentile, the 81^{st}, 82^{nd}, 83^{rd}, 84^{th}, 85^{th}, 86^{th}, 87^{th}, 88^{th}, 89^{th}, 89^{th}, 90^{th}, 91^{st}, 92^{nd}, 93^{rd}, 94^{th}, 95^{th}, 96^{th}, 97^{th}, 98^{th}, or 99^{th} percentile of cortisone in a sample from a normal subject or of cortisone in a lab reference, although the levels may differ for blood versus urine, for example, because of the presence or absence of certain proteins or other components. The subject with a disorder related to HPA axis function may have a baseline saliva, urine, blood, serum, or plasma cortisone level of greater than a concentration between 0.5 pg/mL and 10 pg/mL, between 1 pg/mL and 9 pg/mL, between 2 pg/mL and 8 pg/mL, between 3 pg/mL and 7 pg/mL, between 4 pg/mL and 6 pg/mL, between 2 pg/mL and 4 pg/mL, or between 1 pg/mL and 4 pg/mL. The subject with a disorder related to HPA axis function may have a baseline saliva, urine, blood, serum, or plasma cortisone level of greater than 0.5 pg/mL, greater than 2 pg/mL, greater than 2.5 pg/mL, greater than 2.75 pg/mL, greater than 3 pg/mL, greater than 3.5 pg/mL, greater than 4.0 pg/mL, greater than 4.5 pg/mL, greater than 5 pg/mL, greater than 5.5 pg/mL, greater than 6 pg/mL, greater than 6.5 pg/mL, greater than 7 pg/mL, greater than 7.5 pg/mL, greater than 8 pg/mL, greater than 8.5 pg/mL, greater than 9 pg/mL, or greater than 9.5 pg/mL. The level may be determined against a lab reference. The saliva, urine, blood, serum, or plasma cortisone level may be measured at anytime; for example, in the morning, afternoon, or evening of any given day.

The cortisone may be a hepatic metabolite of cortisone. The hepatic metabolite of cortisone may be tetrahydrocortisone, cortolone, cortolonic acid, or a combination thereof. In a urine sample, the hepatic metabolite of cortisone may measure about 10%, 11%, 12%, 13%, 14%, 15%, 16%, 17%, 18%, 19%, 20%, 21%, 22%, 23%, 24%, 25%, 26%, 27%, 28%, 29%, or about 30% of total glucocorticoids. These amounts may include the level of the hepatic metabolite of cortisol. Tetrahydro metabolites may represent about 60%, 61%, 62%, 63%, 64%, 65%, 66%, 67%, 68%, 69%, 70%, 71%, 72%, 73%, 74%, 75%, 76%, 77%, 78%, 79%, or about 80% of total glucocorticoids. Cortisone may measure about 1%, 2%, 3%, 4%, 5%, 6%, 7%, 8%, 9%, or 10% of total glucocorticoids in a urine sample. Cortisol may measure about 1%, 2%, 3%, 4%, 5%, 6%, 7%, 8%, 9%, or 10% of total glucocorticoids in a urine sample.

The cortisone level may be measured by mass spectrometry or immunoassay in a laboratory of a commercial provider of such services. One such provider is Quest Diagnostics, with corporate headquarters at 3 Giralda Farms, Madison, N.J. 07940, United States of America. Another provider may be a bioanalytical lab such as Pharmaceutical Product Development, Inc., or “PPD.” Depending on the provider of the service and the kind of immunoassay performed, the lab reference range or standard may differ from that of another provider or immunoassay. The cortisone may be measured by mass spectrometry.

(d) Corticotrohpin Releasing Hormone (CRH)

CRH is a 41-amino acid peptide derived from a 191-amino acid preprohormone. CRH is secreted by the paraventricular nucleus (PVN) of the hypothalamus in response to stress. In addition to being produced in the hypothalamus, CRH is also synthesized in peripheral tissues, such as T lymphocytes, and is highly expressed in the placenta.

The subject may have baseline copeptin levels above the 60^{th} percentile, the 70^{th} percentile, the 80^{th} percentile, the 81^{st}, 82^{nd}, 83^{rd}, 84^{th}, 85^{th}, 86^{th}, 87^{th}, 88^{th}, 89^{th}, 90^{th}, 91^{st}, 92^{nd}, 93^{rd}, 94^{th}, 95^{th}, 96^{th}, 97^{th}, 98^{th}, or 99^{th} percentile of CRH in a sample from a normal subject or of CRH in a lab reference. The subject with a disorder related to HPA axis function may have a baseline urine, saliva, blood, serum, or plasma CRH level of greater than a concentration between 0.5 pg/mL and 10 pg/mL, between 1 pg/mL and 9 pg/mL, between 2 pg/mL and 8 pg/mL, between 3 pg/mL and 7 pg/mL, between 4 pg/mL and 6 pg/mL, between 2 pg/mL and 4 pg/mL, or between 1 pg/mL and 4 pg/mL. The subject with a disorder related to HPA axis function may have a baseline blood, serum, or plasma CRH level of greater than 0.5 pg/mL, greater than 1 pg/mL, greater than 2 pg/mL, greater than 2.5 pg/mL, greater than 2.75 pg/mL, greater than 3 pg/mL, greater than 3.5 pg/mL, greater than 4.0 pg/mL, greater than 4.5 pg/mL, greater than 5 pg/mL, greater than 5.5 pg/mL, greater than 6 pg/mL, greater than 6.5 pg/mL, greater than 7 pg/mL, greater than 7.5 pg/mL, greater than 8 pg/mL, greater than 8.5 pg/mL, greater than 9 pg/mL, or greater than 9.5 pg/mL. The level may be determined against a lab reference. The urine, saliva, blood, serum, or plasma CRH level may be measured at anytime; for example, in the morning, afternoon, or evening of any given day.

The CRH level may be measured by immunoassay in a laboratory of a commercial provider of such services. One such provider is Quest Diagnostics, with corporate headquarters at 3 Giralda Farms, Madison, N.J. 07940, United States of America. Depending on the provider of the service and the kind of immunoassay performed, the lab reference range or standard may differ from that of another provider or immunoassay.

(e) Adrenocorticotrophin Hormone (ACTH)

ACTH is a polypeptide tropic hormone produced and secreted by the anterior pituitary gland. As described above, it is a component of the HPA axis. ACTH increases production and release of corticosteroids and cortisol from the adrenal cortex.

The subject may have baseline ACTH levels above the 60^{th} percentile, the 70^{th} percentile, the 80^{th} percentile, the 81^{st}, 82^{nd}, 83^{rd}, 84^{th}, 85^{th}, 86^{th}, 87^{th}, 88^{th}, 89th, 90^{th}, 91^{st}, 92^{nd}, 93^{rd}, 94^{th}, 95^{th}, 96^{th}, 97^{th}, 98^{th}, or 99^{th} percentile of ACTH in a sample from a normal subject or of ACTH in a lab reference. The subject with a disorder related to HPA axis function may have a baseline blood, serum, or plasma ACTH level of greater than a concentration between 0.5 pg/mL and 10 pg/mL, between 1 pg/mL and 9 pg/mL, between 2 pg/mL and 8 pg/mL, between 3 pg/mL and 7 pg/mL, between 4 pg/mL and 6 pg/mL, between 2 pg/mL and 4 pg/mL, or between 1 pg/mL and 4 pg/mL. The subject with a disorder related to HPA axis function may have a baseline blood, serum, or plasma ACTH level of greater than 0.5 pg/mL, greater than 2 pg/mL, greater than 2.5 pg/mL, greater than 2.75 pg/mL, greater than 3 pg/mL, greater than 3.5 pg/mL, greater than 4.0 pg/mL, greater than 4.5 pg/mL, greater than 5 pg/mL, greater than 5.5 pg/mL, greater than 6 pg/mL, greater than 6.5 pg/mL, greater than 7 pg/mL, greater than 7.5 pg/mL, greater than 8 pg/mL, greater than 8.5 pg/mL, greater than 9 pg/mL, or greater than 9.5 pg/mL. The level may be determined against a lab reference. The blood, serum, or plasma ACTH level may be measured at anytime; for example, in the morning, afternoon, or evening of any given day. Changes in the level of ACTH may be associated with symptom changes on an MASQ.

The ACTH level may be measured by immunoassay in a laboratory of a commercial provider of such services. One such provider is Quest Diagnostics, with corporate headquarters at 3 Giralda Farms, Madison, N.J. 07940, United States of America. Another provider may be Thermo Fisher Scientific, Inc or Abbott Laboratories. Commercially available assay kits may be available from Thermo Fisher Scientific, Inc or Abbott Laboratories. Depending on the provider of the service and the kind of immunoassay performed, the lab reference range or standard may differ from that of another provider or immunoassay.

(f) Neurophysin II

Neurophysin II is a carrier protein that binds vasopressin. It is generated from preprovasopressin.

The subject may have neurophysin II levels above the 60^{th} percentile, the 70^{th} percentile, the 80^{th} percentile, the 81^{st}, 82^{nd}, 83^{rd}, 84^{th}, 85^{th}, 86^{th}, 87^{th}, 88^{th}, 89^{th}, 90^{th}, 91^{st}, 92^{nd}, 93^{rd}, 94^{th}, 95^{th}, 96^{th}, 97^{th}, 98^{th}, or 99^{th} percentile of neurophysin II in a sample from a normal subject or of neurophysin II in a lab reference. The subject with a disorder related to HPA axis function may have a baseline blood, serum, or plasma neurophysin II level of greater than a concentration between 1.0 ng/mL and 8.0 ng/mL, between 2 ng/mL and 7 ng/mL, between 3 ng/mL and 6 ng/mL, between 3 ng/mL and 8 ng/mL, 1.5 ng/mL and 6 ng/mL, between 2 ng/mL and 5 ng/mL, or between 3 ng/mL and 5 ng/mL. The subject with a disorder related to HPA axis function may have a baseline blood, serum, or plasma neurophysin II level of greater than 1.5 ng/mL, greater than 2 ng/mL, greater than 2.5 ng/mL, greater than 2.75 ng/mL, greater than 3 ng/mL, greater than 4 ng/mL, greater than 5 ng/mL, greater than 6 ng/mL, greater than 7 ng/mL, or greater than 7.5 ng/mL. The level may be determined against a lab reference. The blood, serum, or plasma neurophysin II level may be measured at anytime; for example, in the morning, afternoon, or evening of any given day.

The subject may have a baseline blood, serum, or plasma neurophysin II level of greater than 2.0 ng/mL, 2.25 ng/mL, 2.50 ng/mL, 2.75 ng/mL, or 3.0 ng/mL as measured using a commercially available immunoassay service or kit. For example, the neurophysin II level may be measured by immunoassay in a laboratory of a commercial provider of such services. Depending on the provider of the service and the kind of immunoassay performed, the lab reference range or standard may differ from that of another provider or immunoassay. The level of blood, serum, or plasma neurophysin II may be expressed in pM.

(g) Glucocorticoids

Glucocorticoids (GCs) may affect renal development and function in fetal and mature kidneys by influencing the cardiovascular system, by their effects on glomerular and tubular function, or a combination of both. Excess GCs due to endogenous GC overproduction in Cushing's syndrome or exogenous GC administration can result in hypertension and cause increased cardiac output, total peripheral resistance and renal blood flow. Glucocorticoids may include tetrahydro metabolites, which may result from cortisol and cortisone being metabolized in the liver by the action of 5-steroid reductases. Glucocorticoids may include cortols, cortolone, cortolic acid, and cortolonic acid. Glucocorticoids may also include cortisol, cortisone, alpha-tetrahydrocortisol, beta-tetrahydrocortisol, and tetrahydrocortisone.

A subject with hypercortisolemia may have increased excretion of cortisol in their urine. This excretion can be compared to creatinine excretion, which may be fairly constant in subjects with normal renal function. The urine glucocorticoid:creatinine ratio of a subject having a disorder related to HPA axis dysfunction may be greater than 2.5, 2.6, 2.7, 2.8, 2.9, 3.0, 3.1, 3.2, 3.3, 3.4. 3.41, 3.42, 3.43, 3.44, 3.45, 3.46, 3.47, 3.48, 3.49, 3.5, 3.6, 3.7, 3.8, 3.9, 4, 5, 6, 7, 8, 9, 10, 10.1, 10.2, 10.3, 10.4, 10.5, 10.6, 10.7, 10.8, 10.9, 11, 11.1, 11.2, 11.3, 11.4, 11.5, 11.55, 11.6, 11.65, 11.7, 11.8, 11.9, 12, 13, 14, 15, 16, 17, 18, 19, or 20.

The glucocorticoid and creatinine levels may be measured by mass spectrometry or immunoassay in a laboratory of a commercial provider of such services. One such provider may be a bioanalytical lab such as Pharmaceutical Product Development, Inc., or “PPD.” Depending on the provider of the service and the kind of mass spectrometry or immunoassay performed, the lab reference range or standard may differ from that of another provider, mass spectrometry method or immunoassay.

d. Subject

The method for identifying an HPA marker can be applied to a sample from a subject. The subject may be human. The subject may be in need thereof. For example, the subject may be diagnosed with a disorder related to HPA axis dysfunction. This diagnosis may be made prior to, during, or after using the methods described herein. The HPA axis dysfunction may drive cortisol elevations that, if unusually extended, can become harmful and result in the disorder. Kaltas and Chrousos (2007), Handbook of psychophysiology, pp. 303-318, New York: Cambridge University Press; and Lovallo and Thomas (2000) Handbook of psychophysiology, pp. 342-367, Cambridge, UK: Cambridge University Press. For example, prolonged cortisol elevations may lead to tissue catabolism, decreased immune function, and neuropsychological effects such as lethargy and disrupted emotional functioning. Side effects of hypercortisolism are similar to symptoms of depression. See Guguis et al., 1990, Biological Psychiatry, 27, 1156-1164. The HPA axis dysfunction may be a deficit of HPA drive or a disrupted diurnal pattern. The subject may be afflicted with one or more other HPA axis dysregulation-related disorders which include a mood disorder, an anxiety disorder, a substance-related disorder, Cushing's syndrome or disease, dementia of the Alzheimer's type, mild cognitive impairment due to Alzheimer's disease, osteoporosis, arthritis, diabetes, dyslipidemia, obesity, hypertension, wounds, pain, and glaucoma. The mood disorder may be depression or a major depressive disorder, for example. The anxiety disorder may be a post-traumatic stress disorder, generalized anxiety disorder, or panic disorder, for example. The substance-related disorder may be alcohol dependence or abuse, or drug dependence or abuse, for example.

The subject may be a normal subject. The normal subject may be free of a disorder associated with HPA axis dysfunction. The normal subject may be healthy and free of any disorder or disease.

e. Sample

The sample, in which the HPA marker(s) may be detected in the methods described above, may be any tissue and comprise protein, hormones, nucleic acid, or a combination thereof from the subject. The hormones may be steroid hormones, such as cortisol, ACTH, CRH, cortisone, and cortisol or cortisone metabolites, for example. The nucleic acid may be DNA or RNA. The nucleic acid may be genomic. The sample may be used directly as obtained from the subject or following pretreatment to modify a character of the sample. Pretreatment may include extraction, concentration, inactivation of interfering components, the addition of reagents, or a combination thereof. The sample may be from the subject who has a disorder characterized by HPA axis dysregulation or it may be a control sample.

The HPA axis function marker may be detected in any cell type, tissue, or bodily fluid sample. Such samples of cell types, tissues, and fluid may include sections of tissues such as biopsy and autopsy samples, frozen sections taken for histological purposes, blood, plasma, serum, saliva, sputum, stool, tears, mucus, lymph fluid, ascetic fluid, gynecological fluid, urine, peritoneal fluid, cerebrospinal fluid, a fluid collected by vaginal rinsing, or a fluid collected by vaginal flushing. A tissue or cell type may be provided by removing a sample of cells from an animal, but can also bee accomplished by using previously isolated cells (e.g., isolated by another person, at another time, and/or for another purpose). Archival tissues, such as those having treatment or outcome history, may also be used. Nucleic acid purification may not be necessary. Any sample, such as a urine sample may be a 24-hour collection of the sample from the subject. The sample may be an overnight collection of the sample from the subject. The sample may be collected from the subject in a single void.

(1) Control/Baseline

It may be desirable to include a control, or baseline, sample, or a series of calibrating compositions for use in the methods described above. The control sample may be analyzed concurrently with the sample from the subject as described above. The results obtained from the subject sample can be compared to the results obtained from the control sample. Standard curves may be provided, with which assay results for the biological sample may be compared. Such standard curves present levels of marker as a function of assay units, i.e. fluorescent signal intensity, if a fluorescent label is used. Using samples taken from multiple donors, standard curves can be provided for control levels of the HPA marker(s) in normal tissue, as well as for “at-risk” levels of the HPA marker in tissue taken from donors, who may have one or more of the characteristics set forth above.

The control may be a sample from a normal subject. The control may be a known level of HPA marker, such as a “lab reference,” or the control may be a known range of levels of HPA marker(s), such as a “lab reference range.”

The lab reference range may vary depending on the assay or the assay service provider. For example, the lab reference range for plasma AVP may be between 0.5 pg/mL and 15 pg/mL, between 1 pg/mL and 13.3 pg/mL, between 1 pg/mL and 15 pg/mL, between 2 pg/mL and 14 pg/mL, between 3 pg/mL and 13 pg/mL, between 4 pg/mL and 12 pg/mL, between 5 pg/mL and 11 pg/mL, between 6 pg/mL and 10 pg/mL, between 7 pg/mL and 9 pg/mL or between 11 pg/mL and 13.3 pg/mL.

The lab reference range for copeptin may be between 2 pmol/L and 20 pmol/L, between 3 pmol/mL and 19 pmol/L, between 4 pmol/mL and 18 pmol/mL, between 4.8 pmol/L and 17.4 pmol/L, between 5 pmol/L and 16 pmol/L, between 7 pmol/L and 14 pmol/L, between 8 pmol/L and 11 pmol/L, between 9 pmol/L and 10 pmol/L, between 10 pmol/L and 17.4 pmol/L, between 13 pmol/L and 17.4 pmol/L or between 14 pmol/L and 17.4 pmol/L.

The lab reference range for copeptin in a female subject may be between 2 pmol/L and 20 pmol/L, between 3 pmol/mL and 19 pmol/L, between 4 pmol/mL and 18 pmol/mL, 4.8 pmol/L and 14 pmol/L, between 5 pmol/L and 14 pmol/L, between 8 pmol/L and 14 pmol/L, between 9 pmol/L and 14 pmol/L, between 10 pmol/L and 14 pmol/L, between 11 pmol/L and 14 pmol/L, between 12 pmol/L and 14 pmol/L, or between 4 pmol/L and 9 pmol/L. The lab reference range for copeptin in a female may be between 4.8 pmol/L and 12.9 pmol/L.

The lab reference range for copeptin in a male subject may be between 4.8 pmol/L and 20 pmol/L, between 5 pmol/L and 20 pmol/L, between 8 pmol/L and 15 pmol/L, between 15 pmol/L and 25 pmol/L, between 16 pmol/L and 24 pmol/L, between 17 pmol/L and 23 pmol/L, between 18 pmol/L and 22 pmol/L, or between 19 pmol/L and 21 pmol/L. The lab reference range for copeptin in a female may be between 4.8 pmol/L and 19.1 pmol/L.

The lab reference range for copeptin may be between 0.5 pg/mL and 10 pg/mL, between 1 pg/mL and 9 pg/mL, between 2 pg/mL and 8 pg/mL, between 3 pg/mL and 7 pg/mL, between 4 pg/mL and 6 pg/mL, between 2 pg/mL and 4 pg/mL, or between 1 pg/mL and 4 pg/mL. The lab reference range may be for any subject, male or female.

The lab reference range for cortisol nmol/L:creatinine mmol/L ratio may be between 1 and 20, 2 and 19, 3 and 18, 4 and 17, 5 and 16, 6 and 15, 7 and 14, 8 and 13, 9 and 12, or 10 and 11. See, for example, Reynolds, et al., “Establishing a reference range for urine cortisol:creatinine ratio,” Endocrine Abstracts (2007)13, P270.

Any of the above-described non-genomic markers may be measured by mass spectrometry or immunoassay in a laboratory of a commercial provider of such services. One such provider is Quest Diagnostics, with corporate headquarters at 3 Giralda Farms, Madison, N.J. 07940, United States of America. Another provider may be Thermo Fisher Scientific, Inc. Commercially available assay kits may be available from Thermo Fisher Scientific, Inc., Perkin Elmer, Abbott Laboratories or Ortho Diagnostics. Depending on the provider of the service and the kind of mass spectrometry or immunoassay performed, the lab reference range or standard may differ from that of another provider, mass spectrometry method or immunoassay.

f. Detection

The HPA marker may be detected in a sample. Many methods are available for detecting a marker in a subject or in a sample obtained from the subject and may be used in conjunction with the herein described methods. The methods include various immunoassays, SNP genotyping, exonuclease-resistant nucleotide detection, solution-based methods, genetic bit analyses, primer guided nucleotide incorporation, allele specific hybridization, and other techniques. Any method of detecting a marker may use a labeled antibody, protein, or oligonucleotide.

The presence or amount of an HPA marker in a sample may be readily determined by, for example, mass spectrometry, immunoassays or immunohistochemistry (e.g. with sections from tissue biopsies) using antibodies (monoclonal or polyclonal) or fragments thereof against the HPA marker. Anti-HPA marker antibodies and fragments thereof can be produced by methods well known in the art. Other methods of detection include those described in, for example, U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety.

(1) Immunoassay

HPA markers, peptides thereof, or combinations thereof, may be analyzed using an immunoassay. The presence or amount of the HPA marker can be determined using antibodies and detecting specific binding to the HPA marker. For example, the antibody, or fragment thereof, may specifically bind to copeptin or AVP.

Any immunoassay may be utilized. The immunoassay may be an enzyme-linked immunoassay (ELISA), radioimmunoassay (RIA), a competitive inhibition assay, such as forward or reverse competitive inhibition assays, a fluorescence polarization assay, or a competitive binding assay, for example. The ELISA may be a sandwich ELISA. Specific immunological binding of the antibody to the HPA marker can be detected via direct labels, such as fluorescent or luminescent tags, metals and radionuclides attached to the antibody or via indirect labels, such as alkaline phosphatase or horseradish peroxidase.

The use of immobilized antibodies or fragments thereof may be incorporated into the immunoassay. The antibodies may be immobilized onto a variety of supports, such as magnetic or chromatographic matrix particles, the surface of an assay plate (such as microtiter wells), pieces of a solid substrate material, and the like. An assay strip can be prepared by coating the antibody or plurality of antibodies in an array on a solid support. This strip can then be dipped into the test biological sample and then processed quickly through washes and detection steps to generate a measurable signal, such as a colored spot.

(a) Sandwich ELISA

The Sandwich ELISA measures the amount of antigen between two layers of antibodies (i.e. capture and a detection antibody). The HPA marker to be measured may contain at least two antigenic sites capable of binding to antibody. Either monoclonal or polyclonal antibodies may be used as the capture and detection antibodies in the sandwich ELISA.

Generally, at least two antibodies are employed to separate and quantify the HPA marker in a test sample. More specifically, the at least two antibodies bind to certain epitopes of the HPA marker forming an immune complex which is referred to as a “sandwich”. One or more antibodies can be used to capture the HPA marker in the test sample (these antibodies are frequently referred to as a “capture” antibody or “capture” antibodies) and one or more antibodies are used to bind a detectable (namely, quantifiable) label to the sandwich (these antibodies are frequently referred to as the “detection” antibody or “detection” antibodies). In a sandwich assay, both antibodies binding to their epitope may not be diminished by the binding of any other antibody in the assay to its respective epitope. In other words, antibodies may be selected so that the one or more first antibodies brought into contact with a test sample suspected of containing the HPA marker do not bind to all or part of an epitope recognized by the second or subsequent antibodies, thereby interfering with the ability of the one or more second detection antibodies to bind to the HPA marker.

The antibodies may be used as a first antibody in said immunoassay. Preferably, the antibody immunospecifically binds to epitopes comprising at least three contiguous (3) amino acids of the HPA marker. In addition to the antibodies of the present invention, said immunoassay may comprise a second antibody that immunospecifically binds to epitopes having an amino acid sequence comprising at least three contiguous (3) amino acids of the HPA marker, wherein the contiguous (3) amino acids to which the second antibody binds is different from the contiguous (3) amino acids to which the first antibody binds.

In a preferred embodiment, a test sample suspected of containing an HPA marker can be contacted with at least one first capture antibody (or antibodies) and at least one second detection antibodies either simultaneously or sequentially. In the sandwich assay format, a test sample suspected of containing the HPA marker is first brought into contact with the at least one first capture antibody that specifically binds to a particular epitope under conditions which allow the formation of a first antibody-HPA marker complex. If more than one capture antibody is used, a first multiple capture antibody-HPA marker complex is formed. In a sandwich assay, the antibodies, preferably, the at least one capture antibody, are used in molar excess amounts of the maximum amount of HPA marker expected in the test sample. For example, from about 5 μg/ml to about 1 mg/ml of antibody per ml of microparticle coating buffer may be used.

Optionally, prior to contacting the test sample with the at least one first capture antibody, the at least one first capture antibody can be bound to a solid support which facilitates the separation the first antibody-HPA marker complex from the test sample. Any solid support known in the art can be used, including but not limited to, solid supports made out of polymeric materials in the forms of wells, tubes or beads. The antibody (or antibodies) can be bound to the solid support by adsorption, by covalent bonding using a chemical coupling agent or by other means known in the art, provided that such binding does not interfere with the ability of the antibody to bind HPA marker. Moreover, if necessary, the solid support can be derivatized to allow reactivity with various functional groups on the antibody. Such derivatization requires the use of certain coupling agents such as, but not limited to, maleic anhydride, N-hydroxysuccinimide and 1-ethyl-3-(3-dimethylaminopropyl)carbodiimide.

After the test sample suspected of containing HPA marker is brought into contact with the at least one first capture antibody, the test sample is incubated in order to allow for the formation of a first capture antibody (or multiple antibody)-HPA complex. The incubation can be carried out at a pH of from about 4.5 to about 10.0, at a temperature of from about 2° C. to about 45° C., and for a period from at least about one (1) minute to about eighteen (18) hours, from about 2-6 minutes, or from about 3-4 minutes.

After formation of the first/multiple capture antibody-HPA marker complex, the complex is then contacted with at least one second detection antibody (under conditions which allow for the formation of a first/multiple antibody-HPA marker-second antibody complex). If the first antibody-HPA marker complex is contacted with more than one detection antibody, then a first/multiple capture antibody-HPA marker-multiple antibody detection complex is formed. As with first antibody, when the at least second (and subsequent) antibody is brought into contact with the first antibody-HPA marker complex, a period of incubation under conditions similar to those described above is required for the formation of the first/multiple antibody-HPA marker-second/multiple antibody complex. Preferably, at least one second antibody contains a detectable label. The detectable label can be bound to the at least one second antibody prior to, simultaneously with or after the formation of the first/multiple antibody-HPA marker-second/multiple antibody complex. Any detectable label known in the art can be used.

(b) Forward Competitive Inhibition

In a forward competitive format, an aliquot of labeled HPA marker of a known concentration is used to compete with HPA marker in a test sample for binding to HPA marker antibody.

In a forward competition assay, an immobilized antibody can either be sequentially or simultaneously contacted with the test sample and a labeled HPA marker. The HPA marker can be labeled with any detectable label in connection with the anti-HPA marker antibodies. In this assay, the antibody can be immobilized on to a solid support. Alternatively, the antibody can be coupled to an antibody, such as an antispecies antibody, that has been immobilized on to a solid support, such as a microparticle.

The labeled HPA marker, the test sample and the antibody are incubated under conditions similar to those described above in connection with the sandwich assay format. Two different species of antibody-HPA marker complexes may then be generated. Specifically, one of the antibody-HPA marker complexes generated contains a detectable label while the other antibody-HPA marker complex does not contain a detectable label. The antibody-HPA marker complex can be, but does not have to be, separated from the remainder of the test sample prior to quantification of the detectable label. Regardless of whether the antibody-marker complex is separated from the remainder of the test sample, the amount of detectable label in the antibody-HPA marker complex is then quantified. The concentration of HPA marker in the test sample can then be determined by comparing the quantity of detectable label in the antibody-HPA marker complex to a standard curve. The standard curve can be generated using serial dilutions of HPA marker of known concentration, by mass spectrometry, gravimetrically and by other techniques known in the art.

The antibody-HPA marker complex can be separated from the test sample by binding the antibody to a solid support, such as the solid supports discussed above in connection with the sandwich assay format, and then removing the remainder of the test sample from contact with the solid support.

(c) Reverse Competition Assay

In a reverse competition assay, an immobilized HPA marker can either be sequentially or simultaneously contacted with a test sample and at least one labeled antibody. Preferably, the antibody specifically binds to an epitope having an amino acid sequence comprising at least three contiguous (3) amino acids of the HPA marker. The HPA marker can be bound to a solid support, such as the solid supports discussed above in connection with the sandwich assay format.

The immobilized HPA marker, test sample and at least one labeled antibody are incubated under conditions similar to those described above in connection with the sandwich assay format. Two different species HPA marker complexes are then generated. Specifically, one of the HPA marker-antibody complexes generated is immobilized and contains a detectable label while the other HPA marker-antibody complex is not immobilized and contains a detectable label. The non-immobilized HPA marker-antibody complex and the remainder of the test sample are removed from the presence of the immobilized HPA marker-antibody complex through techniques known in the art, such as washing. Once the non-immobilized HPA marker antibody complex is removed, the amount of detectable label in the immobilized HPA marker-antibody complex is then quantified. The concentration of HPA marker in the test sample can then be determined by comparing the quantity of detectable label in the HPA marker-complex to a standard curve. The standard curve can be generated using serial dilutions of HPA marker of known concentration, by mass spectrometry, gravimetrically and by other techniques known in the art.

(d) Fluorescence Polarization

In a fluorescence polarization assay, an antibody or functionally active fragment thereof may first contacted with an unlabeled test sample suspected of containing HPA marker to form an unlabeled HPA marker-antibody complex. The unlabeled HPA marker-antibody complex is then contacted with a fluorescently labeled HPA marker. The labeled HPA marker competes with any unlabeled HPA marker in the test sample for binding to the antibody or functionally active fragment thereof. The amount of labeled HPA marker-antibody complex formed is determined and the amount of HPA marker in the test sample determined via use of a standard curve.

The antibody used in a fluorescence polarization assay may specifically bind to an epitope having an amino acid sequence comprising at least three (3) amino acids HPA marker.

The antibody, labeled HPA marker and test sample and at least one labeled antibody may be incubated under conditions similar to those described above in connection with the sandwich immunoassay.

Alternatively, an antibody or functionally active fragment thereof may be simultaneously contacted with a fluorescently labeled HPA marker and an unlabeled test sample suspected of containing HPA marker to form both labeled HPA marker-antibody complexes and unlabeled HPA marker-antibody complexes. The amount of labeled HPA marker-antibody complex formed is determined and the amount of HPA marker in the test sample determined via use of a standard curve.

Alternatively, an antibody or functionally active fragment thereof is first contacted with a fluorescently labeled HPA marker thereof to form a labeled HPA marker-antibody complex. The labeled HPA marker-antibody complex is then contacted with an unlabeled test sample suspected of containing HPA marker. Any unlabeled HPA marker in the test sample competes with the labeled HPA marker for binding to the antibody or functionally active fragment thereof. The amount of labeled HPA marker-antibody complex formed is determined the amount of HPA marker in the test sample determined via use of a standard curve. The antibody used in this immunoassay may specifically bind to an epitope having an amino acid sequence comprising at least three contiguous (3) amino acids of an HPA marker.

(2) Mass Spectrometry

Mass spectrometry (MS) analysis may be used alone or in combination with other methods. Other methods include immunoassays and those described above to detect specific polynucleotides. The mass spectrometry method may be used to determine the presence and/or quantity of one or more biomarkers. MS analysis may comprise matrix-assisted laser desorption/ionization (MALDI) time-of-flight (TOF) MS analysis, such as, for example, directed-spot MALDI-TOF or liquid chromatography MALDI-TOF mass spectrometry analysis. In some embodiments, the MS analysis comprises electrospray ionization (ESI) MS, such as liquid chromatography (LC) ESI-MS. Mass analysis can be accomplished using commercially-available spectrometers. Methods for utilizing MS analysis, including MALDI-TOF MS and ESI-MS, to detect the presence and quantity of an HPA marker in biological samples may be used. See, for example, U.S. Pat. Nos. 6,925,389; 6,989,100; and 6,890,763 for guidance, each of which is incorporated herein by reference.

(3) Genotyping

(a) SNP Genotyping

Large scale SNP genotyping may include any of dynamic allele-specific hybridization (DASH), microplate array diagonal gel electrophoresis (MADGE), pyrosequencing, oligonucleotide-specific ligation, or various DNA “chip” technologies such as Affymetrix SNP chips. These methods may require amplification of the target genetic region. Amplification may be accomplished via polymerase chain reaction (PCR).

(b) Exonuclease-Resistant Nucleotide

PI-markers may be detected using a specialized exonuclease-resistant nucleotide, as described in U.S. Pat. No. 4,656,127, which is incorporated herein by reference. A primer complementary to the allelic sequence immediately 3′ to the polymorphic site may be permitted to hybridize to a target molecule obtained from the subject. If the polymorphic site on the target molecule contains a nucleotide that is complementary to the particular exonuclease-resistant nucleotide derivative present, then that derivative may be incorporated onto the end of the hybridized primer. Such incorporation may render the primer resistant to exonuclease, and thereby permit its detection. Since the identity of the exonuclease-resistant derivative of the sample may be known, a finding that the primer has become resistant to exonuclease reveals that the nucleotide present in the polymorphic site of the target molecule was complementary to that of the nucleotide derivative used in the reaction. This method may not require the determination of large amounts of extraneous sequence data.

(c) Solution-Based Method

A solution-based method may be used to determine the identity of a PI-marker, as described in PCT Application No. WO91/02087, which is herein incorporated by reference. A primer may be employed that is complementary to allelic sequences immediately 3′ to a polymorphic site. The method may determine the identity of the nucleotide of that site using labeled dideoxynucleotide derivatives that, if complementary to the nucleotide of the polymorphic site, will become incorporated onto the terminus of the primer.

(d) Genetic Bit Analysis

Genetic bit analysis may use mixtures of labeled terminators and a primer that is complementary to the sequence 3′ to a polymorphic site. A labeled terminator may be incorporated, wherein it is determined by and complementary to, the nucleotide present in the polymorphic site of the target molecule being evaluated. The primer or the target molecule may be immobilized to a solid phase.

(e) Primer-Guided Nucleotide Incorporation

A primer-guided nucleotide incorporation procedure may be used to assay for a PI-marker in a nucleic acid, as described in Nyren, P. et al., Anal. Biochem. 208:171-175 (1993), which is herein incorporated by reference. Such a procedure may rely on the incorporation of labeled deoxynucleotides to discriminate between bases at a polymorphic site. In such a format, since the signal is proportional to the number of deoxynucleotides incorporated, polymorphisms that occur in runs of the same nucleotide may result in signals that are proportional to the length of the run.

(f) Allele Specific Hybridization

Allele specific hybridization may be used to detect a PI-marker. This method may use a probe capable of hybridizing to a target allele. The probe may be labeled. A probe may be an oligonucleotide. The target allele may have between 3 and 50 nucleotides around the marker. The target allele may have between 5 and 50, between 10 and 40, between 15 and 40, or between 20 and 30 nucleotides around the marker. A probe may be attached to a solid phase support, e.g., a chip. Oligonucleotides may be bound to a solid support by a variety of processes, including lithography. A chip may comprise more than one allelic variant of a target region of a nucleic acid, e.g., allelic variants of two or more polymorphic regions of a gene.

(g) Other Techniques

Examples of other techniques for detecting alleles include selective oligonucleotide hybridization, selective amplification, or selective primer extension. Oligonucleotide primers may be prepared in which the known mutation or nucleotide difference is placed centrally and then hybridized to target DNA under conditions which permit hybridization if a perfect match is found. Such allele specific oligonucleotide hybridization techniques may be used to test one mutation or polymorphic region per reaction when oligonucleotides are hybridized to PCR amplified target DNA or a number of different mutations or polymorphic regions when the oligonucleotides are attached to the hybridizing membrane and hybridized with labeled target DNA.

Allele specific amplification technology that depends on selective PCR amplification may be used in conjunction with the instant invention. Oligonucleotides used as primers for specific amplification may carry the mutation or polymorphic region of interest in the center of the molecule. Amplification may then depend on differential hybridization, as described in Gibbs et al. (1989) Nucleic Acids Res. 17:2437-2448), which is herein incorporated by reference, or at the extreme 3′ end of one primer where, under appropriate conditions, mismatch can prevent, or reduce polymerase extension.

Direct DNA sequencing, either manual sequencing or automated fluorescent sequencing may detect sequence variation. Another approach is the single-stranded conformation polymorphism assay (SSCP), as described in Orita M, et al. (1989) Proc. Natl. Acad. Sci. USA 86:2766-2770, which is incorporated herein by reference. The fragments that have shifted mobility on SSCP gels may be sequenced to determine the exact nature of the DNA sequence variation. Other approaches based on the detection of mismatches between the two complementary DNA strands include clamped denaturing gel electrophoresis (CDGE), as described in Sheffield V C, et al. (1991) Am. J. Hum. Genet. 49:699-706, which is incorporated herein by reference; heteroduplex analysis (HA), as described in White M B, et al. (1992) Genomics 12:301-306, which is incorporated herein by reference; and chemical mismatch cleavage (CMC) as described in Grompe M, et al., (1989) Proc. Natl. Acad. Sci. USA 86:5855-5892, which is herein incorporated by reference. A review of currently available methods of detecting DNA sequence variation can be found in a review by Grompe (1993), which is incorporated herein by reference. Grompe M (1993) Nature Genetics 5:111-117. Once a mutation is known, an allele specific detection approach such as allele specific oligonucleotide (ASO) hybridization can be utilized to rapidly screen large numbers of other samples for that same mutation. Such a technique can utilize probes that may be labeled with gold nanoparticles to yield a visual color result as described in Elghanian R, et al. (1997) Science 277:1078-1081, which is herein incorporated by reference.

A rapid preliminary analysis to detect polymorphisms in DNA sequences can be performed by looking at a series of Southern blots of DNA cut with one or more restriction enzymes, preferably with a large number of restriction enzymes.

(4) Amplification

Any method of detection may incorporate a step of amplifying the HPA-marker. An HPA-marker may be amplified and then detected. Nucleic acid amplification techniques may include cloning, polymerase chain reaction (PCR), PCR of specific alleles (ASA), ligase chain reaction (LCR), nested polymerase chain reaction, self-sustained sequence replication, transcriptional amplification system, and Q-Beta Replicase, as described in Kwoh, D. Y. et al., 1988, Bio/Technology 6:1197, which is incorporated herein by reference.

Amplification products may be assayed by size analysis, restriction digestion followed by size analysis, detecting specific tagged oligonucleotide oligonucleotide primers in reaction products, allele-specific oligonucleotide (ASO) hybridization, allele specific 5′ exonuclease detection, sequencing, hybridization, or a combination thereof.

PCR-based detection means may include amplification of a plurality of markers simultaneously. PCR primers may be selected to generate PCR products that do not overlap in size and may be analyzed simultaneously. Alternatively, one may amplify different markers with primers that are differentially labeled. Each marker may then be differentially detected. Hybridization-based detection means may allow the differential detection of multiple PCR products in a sample.

g. V_{1B} Antagonist

Provided herein are V_{1B} antagonists for use in methods of treating subjects in need thereof. The V_{1B} antagonist may have the following formula (formula I):

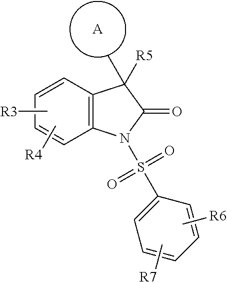

in which A is an aromatic heteromonocyclic ring, where the heterocycles are 5- or 6-membered rings and comprise up to 4 heteroatoms selected from the group consisting of N, O and S, where not more than one of the heteroatoms is an oxygen or sulfur atom, and A may be substituted by radicals R11, R12 and/or R13, where R11, R12 and R13 at each occurrence are selected independently of one another from the group consisting of hydrogen chlorine, bromine, iodine, fluorine, CN, CF₃, OCF₃, NO₂, OH, O—C₁-C₄-alkyl, O-phenyl, O—C₁-C₄-alkylen-phenyl, phenyl, C₁-C₆-alkyl, C₂-C₆-alkenyl, C₂-C₆-alkynyl, NH₂, NH(C₁-C₄-alkyl) and N(C₁-C₄-alkyl)₂, R3 and R4 are selected independently of one another from the group consisting of hydrogen, chlorine, bromine, iodine, fluorine, CN, CF₃, OCF₃, NO₂, OH, O—C₁-C₄-alkyl, O-phenyl, O—C₁-C₄-alkylen-phenyl, phenyl, C₁-C₆-alkyl, C₂-C₆-alkenyl, C₂-C₆-alkynyl, NH₂, NH(C₁-C₄-alkyl) and N(C₁-C₄-alkyl)₂, or R₃ and R₄ are connected to give —CH═CH—CH═CH—, —(CH₂)₄— or —(CH₂)₃—,

### R5 is

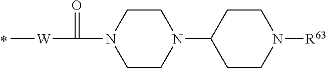

wherein W is selected from the group consisting of NR54, NR54-(C₁-C₄-alkylen) and a bond, R54 is independently selected from the group consisting of hydrogen, C₁-C₆-alkyl, C₂-C₆-alkenyl, C₂-C₆-alkynyl, phenyl and C₁-C₄-alkylen-phenyl, where the phenyl ring may be substituted by up to two radicals R59, R59 is independently selected from the group consisting of hydrogen, chlorine, bromine, iodine, fluorine, CN, CF₃, OCF₃, NO₂, OH, O—C₁-C₄-alkyl, C₁-C₆-alkyl, C₂-C₆-alkenyl, C₂-C₆-alkynyl, NH₂, NH(C₁-C₄-alkyl) and N(C₁-C₄-alkyl)₂, R63 is independently of one another from the group consisting of hydrogen, chlorine, bromine, iodine, fluorine, CN, CF₃, OCF₃, NO₂, OH, O—C₁-C₄-alkyl, O-phenyl, O—C₁-C₄-alkylen-phenyl, phenyl, C₁-C₆-alkyl, C₂-C₆-alkenyl, C₂-C₆-alkynyl, NH₂, NH(C₁-C₄-alkyl) and N(C₁-C₄-alkyl)₂, R6 and R7 are selected independently of one another from the group consisting of hydrogen, chlorine, bromine, iodine, fluorine, CN, CF₃, OCF₃, NO₂, OH, O—C₁-C₄-alkyl atoms, O-phenyl, O—C₁-C₄-alkylen-phenyl, phenyl, C₁-C₆-alkyl, C₂-C₆-alkenyl, C₂-C₆-alkynyl, NH₂, NH(C₁-C₄-alkyl) and N(C₁-C₄-alkyl)₂, and their tautomeric forms, enantiomeric and diastereomeric forms thereof.

A may be an aromatic heteromonocyclic system comprising 1 or 2 heteroatoms, wherein one of the 2 heteroatoms is nitrogen.

A may be pyrimidine, pyridine, pyridazine, pyrazine, thiazole, imidazole, thiophene- or a furan.

The V_{1B} antagonist may be:

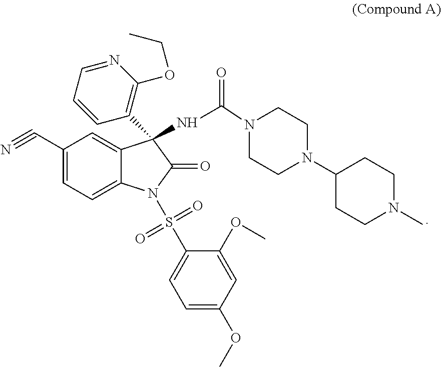 [SMILES: CCOc1ncccc1[C@]1(NC(=O)N2CCN(C3CCN(C)CC3)CC2)C(=O)N(S(=O)(=O)c2ccc(OC)cc2OC)c2ccc(C#N)cc21 ; "."]

The V_{1B} antagonist may be:

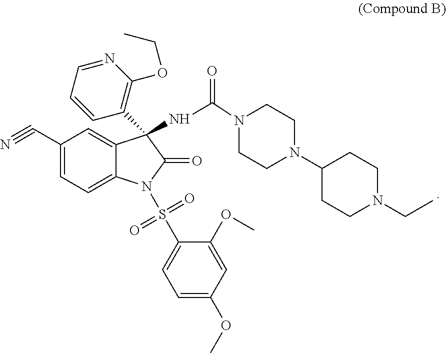 [SMILES: CCOc1ncccc1[C@]1(NC(=O)N2CCN(C3CCN(CC)CC3)CC2)C(=O)N(S(=O)(=O)c2ccc(OC)cc2OC)c2ccc(C#N)cc21 ; "."]

### 4. KIT

Provided herein is a kit, which may be useful for performing any of the methods described herein. The kit may comprise an HPA marker detecting reagent and, optionally, a means for administering the reagent, for example, to a sample. Additionally, the kit may comprise a V_{1B} antagonist as described herein. The kit may comprise protease inhibitor reagents. The kit may comprise pH adjusting reagents, carrier, or a combination thereof. The kit can further comprise instructions for using the kit and conducting the analysis, monitoring, or treatment. The kit may be useful for stabilizing AVP containing sample(s). According to one embodiment, a kit for use in a method of the invention comprises an HPA marker detecting reagent and optionally further components selected from protease inhibitor reagents, pH adjusting reagents, carriers, and a combination thereof. According to a further embodiment, a kit for use in a method of the invention comprises an HPA marker detecting reagent and a V_{1B} antagonist as described herein and optionally further components selected from protease inhibitor reagents, pH adjusting reagents, carriers, and a combination thereof.

The kit may also comprise one or more containers, such as vials, collection tubes, or bottles, with each container containing a separate reagent. For example, a plasma sample that contains AVP may be subjected to one or more protease inhibitors, bioactive carriers, in a container. The kit may further comprise written instructions, which may describe how to stabilize AVP at room temperature, how to perform or interpret an analysis, monitoring, treatment, or method described herein.

The present invention has multiple aspects, illustrated by the following non-limiting examples.

### EXAMPLES

### Example 1

### Plasma AVP Analysis

A randomized, placebo-controlled, double-blind clinical study of the safety and pharmacodynamics of Compound A in subjects with MDD was conducted. Fifty-one subjects with a M.I.N.I.-confirmed primary diagnosis of MDD and a clinical global impression of severity of 3 or 4 were randomized to 800 mg Compound A QD (n=31) or matching placebo (n=20) for 7 days. Subjects were confined at clinical sites from two days prior to initial study drug administration until 24 hours after the last dose of study drug. HPA axis function was thoroughly assessed prior to study drug administration and on the 6th and 7th dosing days. A panel of genetic variants related to HPA axis function was assayed. One stated aim of this study was to define a means to identify MDD subjects more likely to respond to Compound A, prior to Compound A administration.

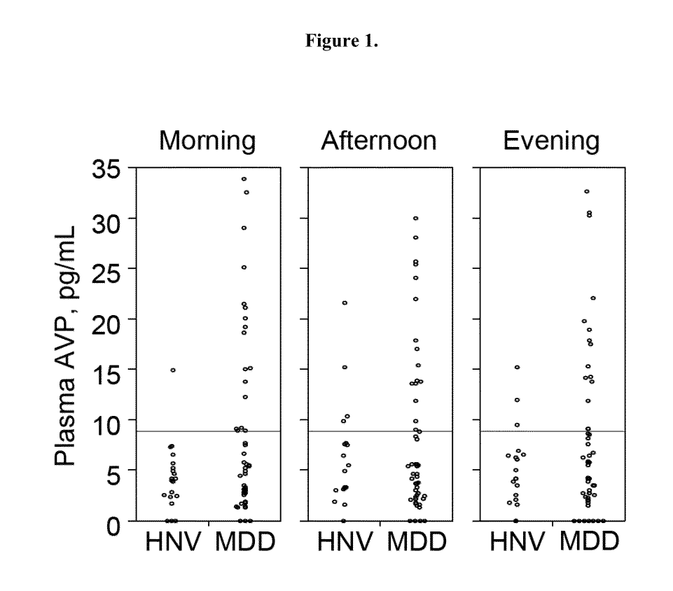

Among MDD subjects, 33% (17/51) showed plasma AVP levels above approximately the 90th percentile (8.6 pg/mL) of the distribution in healthy normal volunteers (HNV). The baseline AVP data in this clinical study are shown with comparable data from another HNV clinical study. These HNV data are consistent with the reported lab reference range (1.0 to 13.3 pg/mL) whereas most of the high AVP MDD subjects showed plasma AVP levels above the lab reference range. See FIG. 1.

AVP was the only parameter initially analyzed as a potential predictor of Compound A response as the daytime plasma level prior to study drug administration, measured at any of three times of day (approximately 8 am, 2 pm, and 10 pm), was the most prominent difference of baseline HPA axis function between MDD subjects in this study and HNV subjects of other studies. MASQ was used as the initial symptom change measure to learn whether high and normal AVP subjects showed differential benefit of Compound A.

Receiver operator characteristic (ROC) analysis was used. In a first ROC, the dependent variable was treatment and the independent variables were the MASQ subscales. This ROC aimed to define MASQ scale score changes that were characteristic of subjects who received Compound A and not characteristic of those who received placebo. Such score changes can be thought to distinguish Compound A responders from Compound A non-responders. Of the five MASQ subscales, General Distress Mixed (GDM) and Depressive (GDD) subscales differentiated Compound A from placebo subjects. See Table 1. This was expected based on ANCOVA results that included statistically significant factors for treatment on those scales but not on Anhedonic Depression (AD), Anxious Arousal (AA) or General Distress Anxiety (GDA). Similar results were obtained when the dependent variables were analyzed as percent, rather than absolute, change from baseline.

TABLE 1

| | | | True | False | |
|---|---|---|---|---|---|
| Scale | X | True Positive | Negative | Positive | False Negative |
| GDM | −0.53 | 24 | 11 | 9 | 7 |
| GDD | −0.58 | 28 | 7 | 13 | 3 |

In a second ROC, the dependent variable was responder status based on a cutoff on GDM of −0.53. GDM was selected as it had better specificity than GDD. The independent variable was baseline afternoon plasma AVP. The optimal cutoff was 8.6 pg/mL. This provided sensitivity of 39% (9/23 Compound A responders included) and specificity of 86% (6 of 7 Compound A non-responders excluded). One Compound A subject was not included in this analysis due to missing baseline afternoon plasma AVP.

An individual's AVP level does not show substantial diurnal variation, thus most subjects defined as high AVP by an afternoon measurement were also so by a morning measurement and similarly for low AVP subjects. One subject who was low AVP by morning measurement and high AVP by afternoon measurement was considered high AVP for all subsequent analyses. One subject who was low AVP by morning measurement and missing the afternoon measurement was considered low AVP for all subsequent analyses.

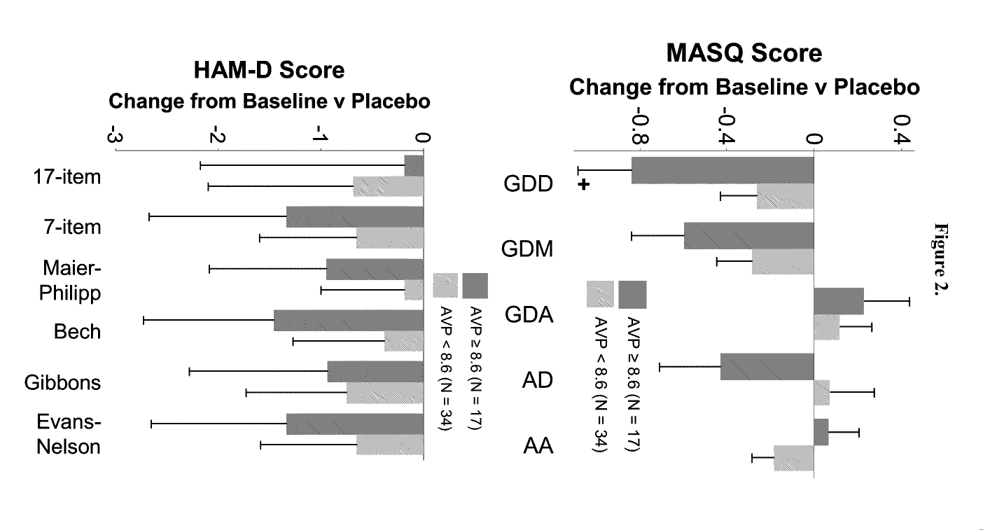

The interaction of AVP (high v normal) and treatment (Compound A v placebo) is shown below. The dependent variables were the MASQ scale scores and the Hamilton Depression Rating Scale (HAM-D)—version score on Day 7. The corresponding scale score on Day −2 was a covariate in each model. Each model included factors for investigator, AVP, treatment and the interaction of AVP and treatment. The last was statistically significant only for MASQ GDD, but there were numerically favorable trends for MASQ GDM and AD as well as the 7-item version of HAM-D. In the figure, bars represent the difference of least squares means between Compound A and placebo in each AVP group and the whiskers represent the standard errors of the differences. MASQ total scores were normalized to a 1 to 5 scale. See FIG. 2.

High AVP and normal AVP MDD subjects, prior to study drug administration, did not differ in most other measures of HPA axis function (plasma ACTH, serum cortisol, urine cortisol and total glucocorticoids, and ACTH and cortisol response to CRH challenge). Normal AVP subjects showed somewhat higher waking saliva cortisol and cortisone, but did not differ from high AVP subjects in afternoon or evening saliva cortisol or cortisone. High and normal AVP MDD subjects did not show any clear differences of symptoms as assessed by total scores on MASQ, HAM-D or the Perceived Stress Scale. The two groups also did not differ in the neuroendocrine effects of Compound A on plasma ACTH, serum cortisol, or urine cortisol and total glucocorticoids. Saliva cortisol and cortisone during Compound A treatment was similar between the two groups, implying a somewhat larger change from prior to study drug administration among normal AVP subjects.

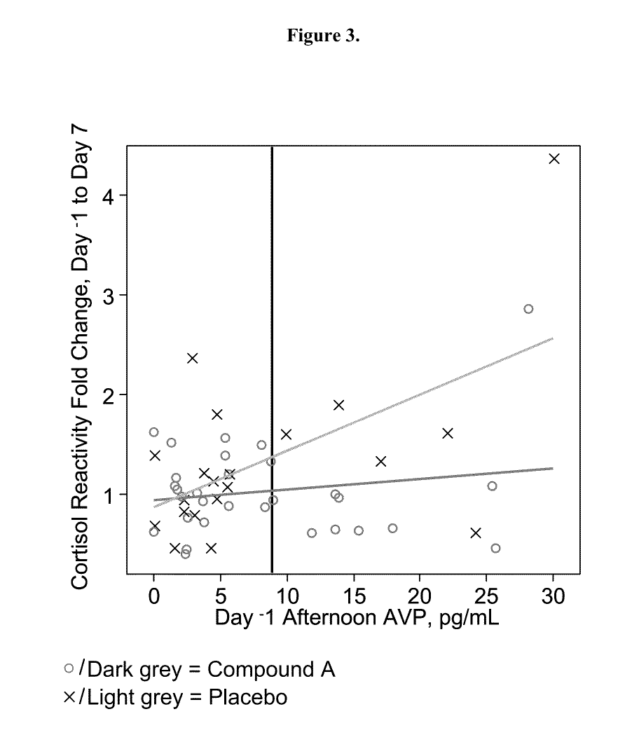

High AVP MDD subjects who received Compound A showed lower physiological response to a stressor (CRH challenge) compared to those who received placebo. In contrast, there was limited or no difference of stress response between normal AVP MDD subjects who received Compound A or placebo. See FIG. 3. High AVP subjects are those for whom attenuation of HPA axis activity is relevant for their physiological response to stress and depressive symptoms, and thus an appropriate population for treatment of MDD using a V_{1B} antagonist.

### Example 2

### Copeptin and AVP-Associated Genetic Markers

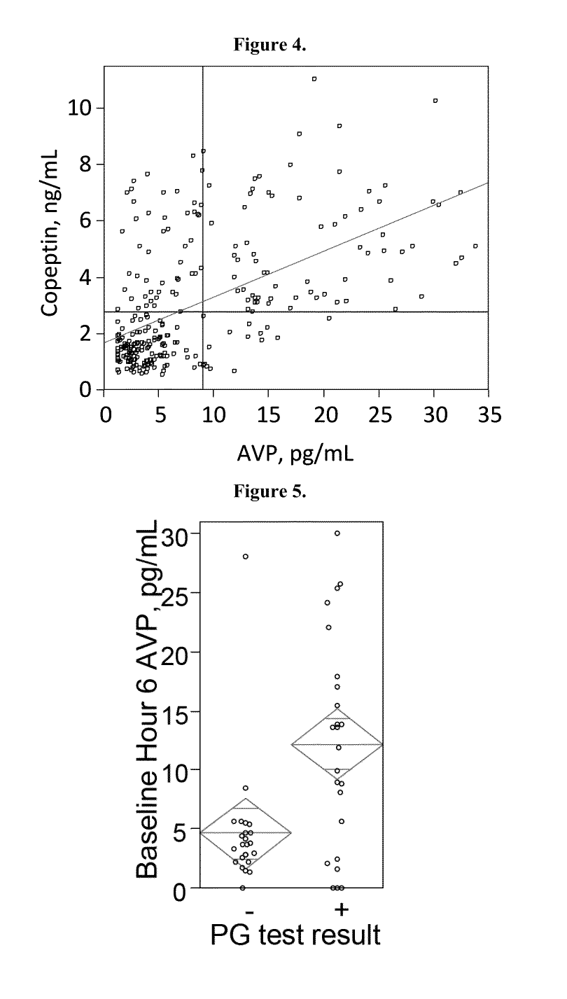

Copeptin is a fragment of AVP precursor protein that has a long half-life in vivo and is stable in plasma ex vivo. In the clinical study described in Example 1, copeptin levels correlated well with AVP levels. The data shown are morning, afternoon and evening levels, prior to study drug initiation and after administration of the sixth daily study drug dose. See FIG. 4.

In addition, association was assessed between a panel of HPA- or depression-related genetic variants and baseline AVP levels prior to study drug administration. A combination of two genotypes, LHPP rs7088418 AA and AKR1D1 rs17169521 GG, appeared to separate high AVP from most normal AVP individuals. See FIG. 5.

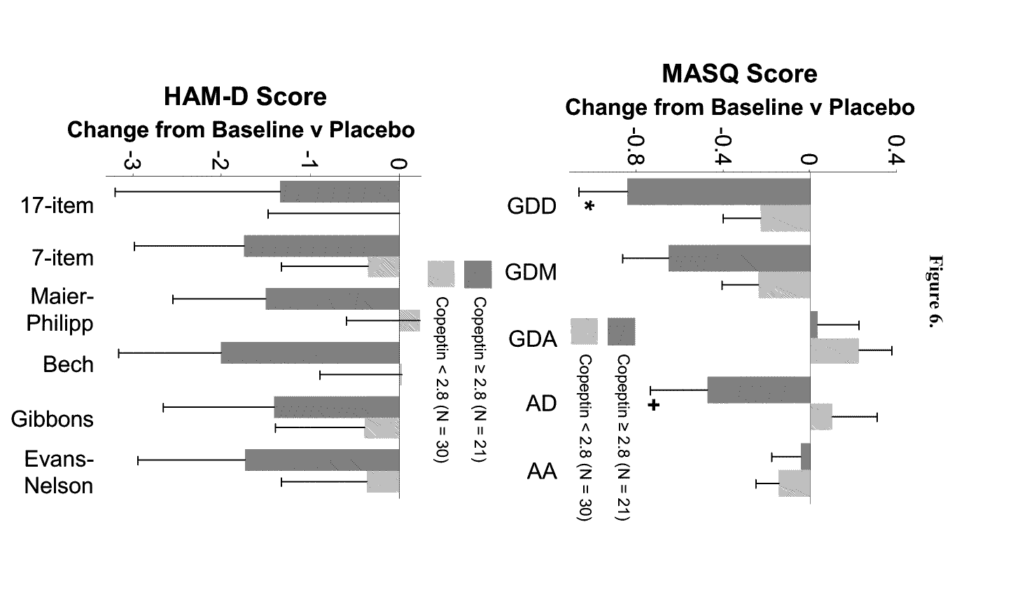

Copeptin (using 2.8 ng/mL as a cutoff for high copeptin) functioned similarly as AVP as a predictor of Compound A-associated depressive symptom changes. Copeptin appeared to be a superior predictor of Compound A-associated changes of HAM-D scores. The difference compared to AVP may be mostly attributed to a single subject who showed a large HAM-D-17 decrease, normal AVP and high copeptin. It is possible that AVP values for this subject, as well as four others who showed normal AVP and high copeptin, were in the normal range due to preanalytic instability of AVP. See FIG. 6.

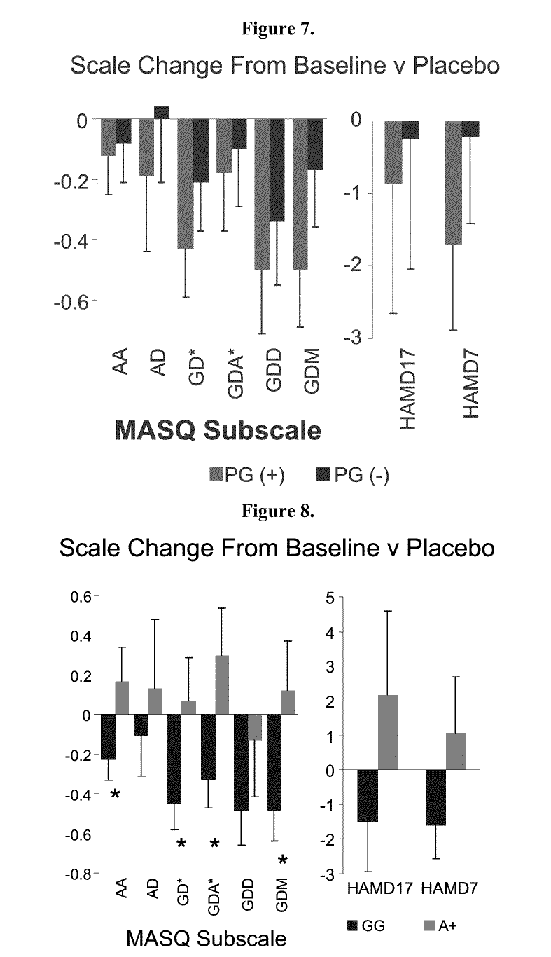

The combination of two genotypes behaved similarly as high AVP as a predictor of depressive symptom changes in the clinical study described in Example 1. PG-positive status is a combination of rs7088418 AA & rs17169521 GG genotypes, and was observed in 24 of 48 (50%) subjects. Data from 3 subjects were missing for one or both genotypes in the multiplex assay used. See FIG. 7.

### Example 3

The same panel of HPA- or depression-related genetic variants was assessed for association with symptom improvement in subjects. Some evidence for association was noted for certain NR3C1 genotypes. In contrast to AVP, copeptin and rs7088418/rs17169521 genotypes that distinguished a subset of subjects who showed more favorable symptom changes on MASQ and HAM-D with Compound A administration, the NR3C1 genotypes distinguished a subset of subjects who showed unfavorable symptom changes on MASQ and HAM-D with Compound A administration. The following data are for rs10482672; similar results were obtained for rs17100236. 36 of 48 (75%) subjects had rs10482672 genotype GG. Data from 3 subjects were missing for rs10482672 in the multiplex assay used. See FIG. 8. AVP levels were not associated with rs10482672 and rs17100236 genotypes.

### Example 4

Cortisol and cortisone are metabolized in the liver mainly to tetrahydro metabolites by the action of 5-steroid reductases. These tetrahydro metabolites are rapidly excreted via the urine and are the most prevalent urine glucocorticoids, generally comprising approximately 70% of total urine glucocorticoids. Unmetabolized cortisol and cortisone are excreted in urine directly from the kidneys and generally comprise approximately 5% of total urine glucocorticoids. Other significant urine glucocorticoids include cortols, cortolone, cortolic acids and cortolonic acid.

In a ROC analysis, the dependent variable was responder status based on a cutoff on GDM of −0.53. The independent variable was baseline amount of urine glucocorticoids (the sum of cortisol, cortisone, alpha-tetrahydrocortisol, beta-tetrahydrocortisol and tetrahydrocortisone) collected during a 24-hour interval, divided by the amount of creatinine in the same urine collection. The optimal cutoff was 3.44 mg glucocorticoids per mg creatinine. This provided a sensitivity of 58% (14/24 Compound A responders included) and specificity of 67% (4 of 6 Compound A non-responders excluded). One Compound A subject was not included in this analysis due to missing urine glucocorticoid data.

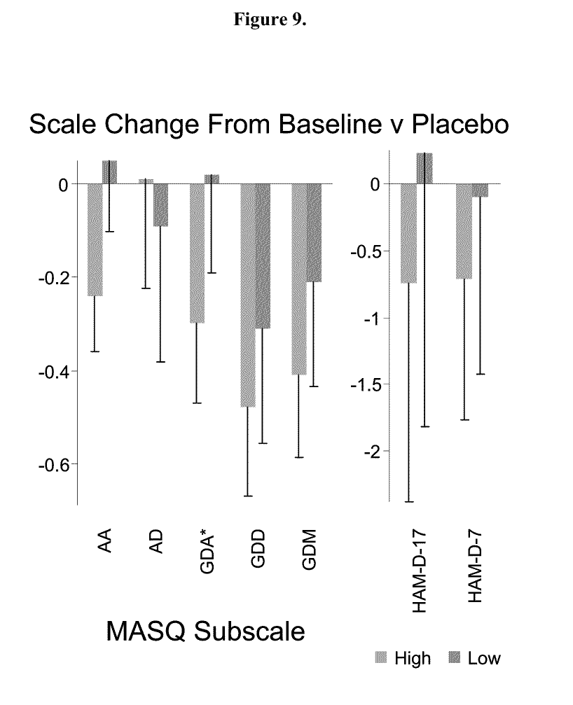

The interaction of urine glucocorticoids divided by creatinine (higher v lower) and treatment (Compound A v placebo) is shown below. The dependent variables were the MASQ scale scores and the Hamilton Depression Rating Scale (HAM-D)-version score on Day 7. In this analysis, three MASQ items pertaining to gastrointestinal symptoms were omitted from GDA, which might compromise the psychometric validity of that specific scale. The corresponding scale score on Day −2 was a covariate in each model. Each model included factors for investigator, AVP, treatment and the interaction of AVP and treatment. The last was not statistically significant for any of the dependent variables, but there were numerically favorable trends for MASQ GDM, GDD, GDA and AA as well as the 17- and 7-item versions of HAM-D. In the figure, bars represent the difference of least squares means between Compound A and placebo in each urine glucocorticoids group and the whiskers represent the standard errors of the differences. MASQ total scores were normalized to a 1 to 5 scale. See FIG. 9.

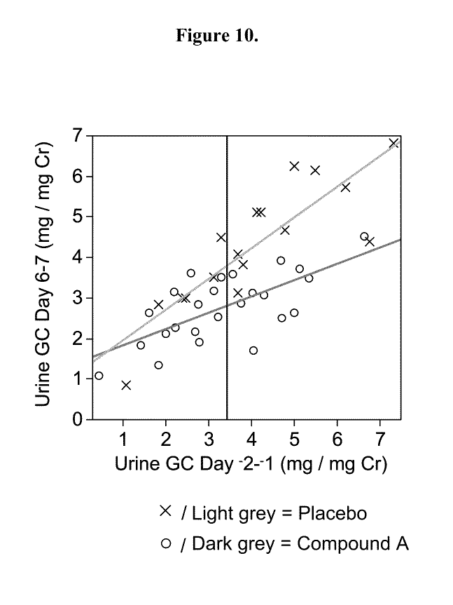

Higher urine glucocorticoids MDD subjects who received Compound A showed larger reductions of urine glucocorticoids compared to those who received placebo. In contrast, there was limited or no difference of reduction of urine glucocorticoids between lower urine glucocorticoids MDD subjects who received Compound A or placebo. See FIG. 10. The same relationship has been observed among healthy adults who received Compound A or placebo. Higher urine glucocorticoids subjects are those in whom Compound A effects larger decreases of tonic HPA axis activity, and thus an appropriate population for treatment of MDD using a V1B antagonist.

Similar results were obtained when the amounts of urine glucocorticoids and creatinine from an overnight (10-hour) collection were used in place of those from a 24-hour collection.

We claim:

1. A method for determining whether a subject is a suitable candidate for treatment with a V_{1B} antagonist and treating a subject identified as a suitable candidate for said treatment, the method comprising the steps of:
(a) providing a biological sample from the subject; and
(b) detecting a hypothalamus-pituitary-adrenal (“HPA”) axis function marker;
wherein the subject has a disorder characterized by HPA axis dysregulation;
wherein the presence of the marker indicates that the subject is a suitable candidate for treatment with a V_{1B} antagonist; and
(c) treating a subject that is identified as a suitable candidate in step (b) with a V_{1B} antagonist.

2. The method of claim 1, wherein the HPA axis function marker is selected from the group consisting of a nucleotide sequence comprising SEQ ID NO:1 (LHPP rs7088418) and a nucleotide sequence comprising SEQ ID NO:2 (AKRID1 rs17169521); a nucleotide sequence comprising an NR3C1 genotype; and combinations thereof.

3. The method of claim 2, wherein the NR3C1 genotype is selected from the group consisting of SEQ ID NO:3 (rs10482672) and SEQ ID NO:4 (rs17100236).

4. The method of claim 1, wherein the marker is detected by is detected by genotyping.

5. The method of claim 4, wherein the genotyping comprises
(a) amplifying a nucleic acid comprising the marker; and
(b) detecting the amplified nucleic acids, thereby detecting the marker.

6. The method of claim 5, wherein the marker is detected by sequencing.

7. A method for determining whether a subject is a suitable candidate for treatment with a V_{1B} antagonist and treating a subject identified as a suitable candidate for said treatment, the method comprising the steps of:
(a) providing a biological sample from the subject; and
(b) detecting a hypothalamus-pituitary-adrenal (“HPA”) axis function marker;
wherein the subject has a disorder characterized by HPA axis dysregulation;
wherein if the HPA axis function marker is present at a level above about the 60^{th} percentile of the distribution of the marker in a normal subject sample, the subject is suitable for treatment with a V_{1B} antagonist; and
(c) treating a subject that is identified as a suitable for treatment in step (b) with a V_{1B} antagonist

8. The method of claim 7, wherein the HPA axis function marker is present at a level greater than a percentile selected from the group consisting of about the 65^{th}, 70^{th}, 75^{th}, 80^{th}, 85^{th}, 90^{th}, and 95^{th} percentile of the distribution of the HPA axis function marker in a normal subject sample.

9. The method of claim 7, wherein the marker is selected from the group consisting of AVP, copeptin, cortisol, cortisone, ACTH, hepatic metabolite of cortisol, hepatic metabolite of cortisone, CRH, and a combination thereof.

10. The method of claim 9, wherein the copeptin is plasma copeptin.

11. The method of claim 9, wherein the AVP is plasma AVP.

12. The method of claim 9, wherein the hepatic metabolite of cortisol is selected from the group consisting of alpha-tetrahydrocortisol, beta-tetrahydrocortisol, alpha-cortol, beta-cortol, alpha-cortolic acid, beta-cortolic acid, and a combination thereof.

13. The method of claim 9, wherein the hepatic metabolite of cortisone is selected from the group consisting of tetrahydrocortisone, cortolone, cortolonic acid, and a combination thereof.

14. The method of claim 1 or 7, wherein the biological sample is selected from the group consisting of a nucleic acid containing sample, serum, plasma, blood, urine, and saliva.

15. The method of claim 14, wherein the biological sample is urine.

16. The method of claim 15, wherein the marker is the sum of urine amounts of cortisol, cortisone, alpha-tetrahydrocortisol, beta-tetrahydrocortisol, and tetrahydrocortisone.

17. The method of claim 15, wherein the marker is the sum of urine amounts of alpha-tetrahydrocortisol, beta-tetrahydrocortisol, and tetrahydrocortisone.

18. The method of claim 16 or 17, wherein the sum of the urine amounts is divided by the amount of creatinine in the same urine sample.

19. The method of claim 16, 17 or 18, wherein the urine sample is a 24-hour collection of urine from the subject.

20. The method of claim 16, 17 or 18, wherein the urine sample is an overnight collection of urine from the subject.

21. The method of claim 16, 17 or 18, wherein the urine sample is collected from the subject in a single void.

22. The method of claim 7, wherein the sample is a plasma sample that contains greater than or equal to 8.6 pg/mL AVP as determined by radioimmunoassay.

23. The method of claim 7, wherein the sample is a plasma sample that contains greater than or equal to 2.8 ng/mL of copeptin as determined by enzyme immunoassay.

24. The method of claim 7, wherein the sample is urine sample that contains a sum of cortisol, cortisone, alpha-tetrahydrocortiso, beta-tetrahydrocortisol and tetrahydrocortisone greater than or equal to 3.44 mg per mg of creatinine.

25. The method of claim 7, wherein the marker is detected by an immunoassay.

26. The method of claim 7, wherein the marker is detected by mass spectrometry.

27. The method of claim 1 or 7, wherein the disorder is selected from the group consisting of Cushing's syndrome, dementia, cognitive impairment, mood disorder, anxiety disorder, substance-related disorder, osteoporosis, arthritis, diabetes, dyslipidemia, obesity, hypertension, pain, glaucoma, and combinations thereof.

28. The method of claim 27, wherein the mood disorder is depression.

29. The method of claim 28, wherein the depression is major depressive disorder.

30. The method of claim 27, wherein the anxiety disorder is selected from the group consisting of post-traumatic stress disorder, generalized anxiety disorder and panic disorder.

31. The method of claim 27, wherein the substance-related disorder is selected from the group consisting of alcohol dependence or abuse, and drug dependence or abuse.

32. The method of claim 27, wherein the dementia is of the Alzheimer's type.

33. The method of claim 27, wherein the cognitive impairment is mild cognitive impairment due to Alzheimer's disease.

34. A method for monitoring a subject's response to treatment with a V_{1B} antagonist, comprising
(a) providing a biological sample from the subject receiving treatment with a V_{1B} antagonist;
(b) detecting a hypothalamus-pituitary-adrenal (“HPA”) axis function marker;
wherein the subject has a disorder characterized by HPA axis dysregulation; and
wherein a greater than 25% change in the level of the marker as compared to a baseline, indicates that the V_{1B} antagonist is useful for treating the subject; and
(c) continuing or discontinuing treatment with the V_{1B} antagonist in the subject based on the change in the level of the marker as compared to baseline detected in step (b).

35. The method of claim 34, wherein the HPA axis function marker is selected from the group consisting of cortisol; cortisone; corticotrophin releasing hormone adrenocorticotrophin hormone (ACTH); hepatic metabolite of cortisol, hepatic metabolite of cortisone, and combinations thereof.

36. The method of claim 35, wherein the hepatic metabolite of cortisone is selected from the group consisting of tetrahydrocortisone, cortolone, cortolonic acid, and a combination thereof.

37. The method of claim 35, wherein the marker is the sum of urine amounts of cortisol, cortisone, alpha-tetrahydrocortisol, beta-tetrahydrocortisol, and tetrahydrocortisone.

38. The method of claim 35, wherein the marker is the sum of urine amounts of alpha-tetrahydrocortisol, beta-tetrahydrocortisol, and tetrahydrocortisone.

39. The method of claim 37 or 38, wherein the sum of the urine amounts is divided by the amount of creatinine in the same urine sample.

40. The method of claim 34, wherein the baseline is cortisol at a level of between 3 pg/mL and 13 pg/mL.

41. The method of claim 34, wherein the baseline of cortisol is determined using enzyme immunoassay.

42. The method of claim 34, wherein the baseline indicates the level of the HPA axis function marker in a sample taken from the subject prior to beginning V1B antagonist therapy.

43. The method of claim 35, wherein the ACTH is plasma ACTH.

44. The method of claim 34, wherein the biological sample is selected from the group consisting of a nucleic acid containing sample, serum, plasma, blood, urine, and saliva.

45. The method of claim 34, wherein the disorder is selected from the group consisting of Cushing's syndrome, dementia, cognitive impairment, mood disorder, anxiety disorder, substance-related disorder, osteoporosis, arthritis, diabetes, dyslipidemia, obesity, hypertension, pain, glaucoma, and combinations thereof.

46. The method of claim 45, wherein the mood disorder is depression.

47. The method of claim 46, wherein the depression is major depressive disorder.

48. The method of claim 45, wherein the anxiety disorder is selected from the group consisting of post-traumatic stress disorder, generalized anxiety disorder and panic disorder.

49. The method of claim 45, wherein the substance-related disorder is selected from the group consisting of alcohol dependence or abuse, and drug dependence or abuse.

50. The method of claim 45, wherein the dementia is of the Alzheimer's type.

51. The method of claim 45, wherein the cognitive impairment is mild cognitive impairment due to Alzheimer's disease.

52. The method of claim 34, wherein the greater than 25% change in the level of the marker is an increased change compared to the baseline.

53. The method of claim 34, wherein the greater than 25% change in the level of the marker is a decreased change compared to the baseline.

54. The method of claim 1, 7, or 34, wherein the V_{1B} antagonist is of formula I: 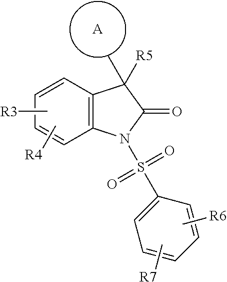
in which A is an aromatic heteromonocyclic ring, where the heterocycles are 5- or 6-membered rings and comprise up to 4 heteroatoms selected from the group consisting of N, O and S, where not more than one of the heteroatoms is an oxygen or sulfur atom, and A may be substituted by radicals R11, R12 and/or R13, where R11, R12 and R13 at each occurrence are selected independently of one another from the group consisting of hydrogen chlorine, bromine, iodine, fluorine, CN, CF₃, OCF₃, NO₂, OH, O—C₁-C₄-alkyl, O-phenyl, O—C₁-C₄-alkylen-phenyl, phenyl, C₁-C₆-alkyl, C₂-C₆-alkenyl, C₂-C₆-alkynyl, NH₂, NH(C₁-C₄-alkyl) and N(C₁-C₄-alkyl)₂, R3 and R4 are selected independently of one another from the group consisting of hydrogen, chlorine, bromine, iodine, fluorine, CN, CF₃, OCF₃, NO₂, OH, O—C₁-C₄-alkyl, O-phenyl, O—C₁-C₄-alkylen-phenyl, phenyl, C₁-C₆-alkyl, C₂-C₆-alkenyl, C₂-C₆-alkynyl, NH₂, NH(C₁-C₄-alkyl) and N(C₁-C₄-alkyl)₂, or R₃ and R₄ are connected to give —CH═CH—CH═CH—, —(CH₂)₄— or —(CH₂)₃—,
R5 is 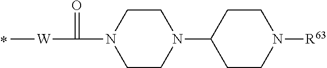
wherein W is selected from the group consisting of NR54, NR54-(C₁-C₄-alkylen) and a bond, R54 is independently selected from the group consisting of hydrogen, C₁-C₆-alkyl, C₂-C₆-alkenyl, C₂-C₆-alkynyl, phenyl and C₁-C₄-alkylen-phenyl, where the phenyl ring may be substituted by up to two radicals R59, R59 is independently selected from the group consisting of hydrogen, chlorine, bromine, iodine, fluorine, CN, CF₃, OCF₃, NO₂, OH, O—C₁-C₄-alkyl, C₁-C₆-alkyl, C₂-C₆-alkenyl, C₂-C₆-alkynyl, NH₂, NH(C₁-C₄-alkyl) and N(C₁-C₄-alkyl)₂, R63 is independently of one another from the group consisting of hydrogen, chlorine, bromine, iodine, fluorine, CN, CF₃, OCF₃, NO₂, OH, O—C₁-C₄-alkyl, O-phenyl, O—C₁-C₄-alkylen-phenyl, phenyl, C₁-C₆-alkyl, C₂-C₆-alkenyl, C₂-C₆-alkynyl, NH₂, NH(C₁-C₄-alkyl) and N(C₁-C₄-alkyl)₂, R6 and R7 are selected independently of one another from the group consisting of hydrogen, chlorine, bromine, iodine, fluorine, CN, CF₃, OCF₃, NO₂, OH, O—C₁-C₄-alkyl atoms, O-phenyl, O—C₁-C₄-alkylen-phenyl, phenyl, C₁-C₆-alkyl, C₂-C₆-alkenyl, C₂-C₆-alkynyl, NH₂, NH(C₁-C₄-alkyl) and N(C₁-C₄-alkyl)₂, and their tautomeric forms, enantiomeric and diastereomeric forms thereof.

55. The method of claim 54, wherein A is an aromatic heteromonocyclic system comprising 1 or 2 heteroatoms, wherein one of the 2 heteroatoms is nitrogen.

56. The method of claim 54, wherein A is selected from the group consisting of pyrimidine, pyridine, pyridazine, pyrazine, thiazole, imidazole, thiophene- and furan.

57. The method of claim 54, wherein the V_{1B} antagonist is:

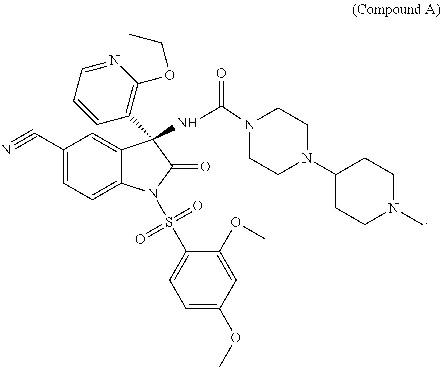 [SMILES: CCOc1ncccc1[C@]1(NC(=O)N2CCN(C3CCN(C)CC3)CC2)C(=O)N(S(=O)(=O)c2ccc(OC)cc2OC)c2ccc(C#N)cc21 ; "."]

58. The method of claim 54, wherein the V_{1B} antagonist is:

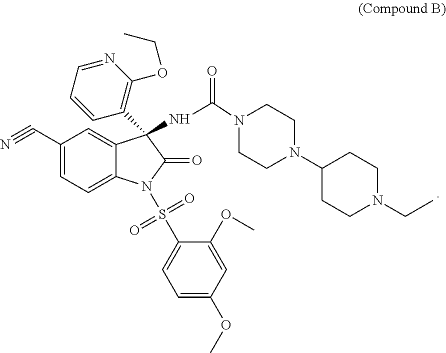 [SMILES: CCOc1ncccc1[C@]1(NC(=O)N2CCN(C3CCN(CC)CC3)CC2)C(=O)N(S(=O)(=O)c2ccc(OC)cc2OC)c2ccc(C#N)cc21 ; "."]

59. The method of claim 1 or 7, wherein the method is used to screen subjects for eligibility for a clinical trial.

60. The method of claim 1 or 7, wherein the method is used to stratify randomization of subjects for a clinical trial.

61. The method of claim 1 or 7, wherein the method is used to stratify analysis of a clinical trial.

62. A kit for stabilizing AVP in a plasma sample, wherein the kit comprises one or more collection tubes comprising one or more protease inhibitors.

63. A kit for assay of AVP in a blood-derived matrix, wherein the kit comprises a collection tube and instructions for stabilizing AVP at room temperature.
